SEQUENCE LISTING (13 sequences)

SEQ ID NO: 1 - DNA, 2101 bp, T trogii
atggccaggttccaatctctcctcaccttcatcaccctctcgctcgttgcctccgtgtacgctgccatcgggccagtcgcagacctcaccatctccaatggtgccgtcagtcccgatggtttctctcggcaggcgatcctggtcaacgacgtcttccccagtcccctcatcacgggtaacaaggccagttgttttctccatcgccgttcaccgcacatgctgacctacttacagggtgatcgcttccaactcaacgtcatcgacaacatgacgaaccacaccatgttgaagtctactagtatcgtgagtacctcggcgtactgtccgatttaccgctcacattgctcctttcagcactggcacggcttcttccaacacggtacgaactgggccgatggccccgccttcgtcaaccagtgccccatttctaccgggcatgcgttcctttacgacttccaggtccctgaccaagctggcgagttgcaaacgatatatggctgacgaacaaagtccggtgctaatagcctactgcaataggtactttctggtaccacagtcacttgtccactcagtactgtgatggtctcaggggtccgattgttgtctatgaccctcaagacccccacaagagcctctacgacgttgatgacggtaatgccattgatactggagtctttgcgaatgctaacatgttttcttgcagactccactgtgatcactctcgcggactggtaccacttagctgccaaagttggctcgcctgtcccgtgagtctatttcgtctggagaatactctatttttgctgacatccagtcatgttctcacaggactgcagacgcgactctcatcaacggcctcggtcgcagcatcgacacgctcaacgccgatttggctgtcatcacggtcacgaagggcaagcggtacgtgtccaacccagaggtcaaaatgcgggtatcctaatgaacgtcatgtagctatcgcttccgccttgtgtccctttcatgcgacccgaaccacgtgttcagtattgacggtcactctctgaccgttatcgaggcagacagcgtgaacctgaagccccagactgtcgactccatccagatcttcgccgcccagcggtactcgtttgtagtaggttggacacggcttcaacaggcgtgcatgtcgctcacccactgtcatagctaaatgcagatcaggatgtgggcaactactggatccgcgccctgcccaactccggaaccaggaacttcgacggtggggtcaactccgccatcctccgctacgacggtgctgcgcctgttgagcccacaacgtctcagacgccgtcgacgaatcctttggtggagtccgccctcactactctcgaaggcaccgctgcagtatgtgatctgcggacgaacaaccgagcgctgtctacctatcgcattgtctagcccggcagcccggctcctggcggtgtcgacctagcgctcaacatggcgtttggcttcgtaagtcgcgcccctgttaagtacacagcacatggctcatcatcgtttcaggccggcggcaaattcaccatcaacggcgcgagctttacccctcccacagtccctgtcctcctgcagatcctgagcggcgcgcagtcggcgcaggacctcctaccctccggaagtgtatactcgctccctgcgaacgcggacattgagatatccctgcccgccaccgccgccgcccctggcttcccgcaccccttccatttgcacggggtacgtctcaccggcccctcctaacgccctcacgctgacctcctattccacccaatagcacaccttcgccgtcgtgcgcagcgcgggctcgtccacgtacaactacgagaacccggtctaccgcgatgtggtcagcaccggctcgcccggggacaacgtcacgatccggttccggacggacaaccccggcccgtggttcctccactgccatatcgacttccacctcgaggcgggctttgcggtcgtcatggccgaggacattcccgaggtcgctgctacgaacccggtcccgcaggcatggtcggacctgtgcccgacttacgacgcgctctcgcctgacgaccagtag

SEQ ID NO: 2 - DNA, 1563 bp, P brumalis
atggcgaggttccagtccctgctctcctacgtcaccctcctctttgtggcgtcagcatacgctgccattggcccggtcaccgacctcactgtcaccgatgctaacatcagccccgatggcttcacccgtgccggtatcgtggtcaacaatgtctttcccgcgccgctcatcactggccagaagggggaccgcttccaactgaatctcgtcaatcagatgagcaaccacacgatgctgaagaccactagtatccactggcatggcttcttccagaagggtactaactgggcggacggccctgcgttcgtgaaccagtgcccaattgctagcggaaactccttcctctatgacttccaggtacccgaccaagcaggtaccttctggtatcacagccacctgtccactcagtactgtgatggtctgaggggtccgttcgtcgtgtacgatcctaccgacccccatctctccctctacgacgttgatgacgactcgaccgtcattacactggcagattggtaccacgttgctgctaggctcggtccgcggttcccgctcggtgctgactcgaccctcatcaacggtctcggtcgtagtaccgctaccccgacggccgacctggctgtcatcagcgtcaccaaggggaagcggtaccggttccgcctcgtatcgatctcttgcgaccccaaccacacgttcagcatcgatggtcacaagctgaccgtcatagaggccgacggtatcagcacccagccggtcactgggattgactccatccagatcttcgctgctcagcgttattcgttcgttctcaccgccgaccaagatgtagacaactactgggtccgtgctaacccgaacttcggcaccaccggtttcgctggcggcattaactcggccatcttgcgttacgatggcgcgccagctgttgagcctaccacgagccaaaccggcacgaacctgcttgtggagactgacctccacccattgagtaccatgcctgtgcccggtctccccactcagggtggtgctgacttcaacctcaacctggcgttcaacttcaatggctcagactttttcatcaacggcgcctccttcgttcccccgactgtcccggtcctgctgcagatcatctccggtgccaactcggcccaggacctcctcccctctggcagcgtctacgcgctcccgtccaactcgtccatcgagctcaccttccctgctactgccgccgctcccggtgccccgcaccccttccacctgcacggtcacgccttcgctgtcgtacgcagcgcaggtagcacggtctataactacgacaaccccgtcttccgcgacgtcgtgagcacgggcacgcccgccgctggagacaatgtcacgatccgcttccagacggacaacccgggcccgtggttcctccactgccacatcgacttccacctcgacgccggctttgcggtcgtgttcgctgaggacctccccgatgtcgtgtccgcgaaccccgtgccgcaggcctggtccgacctctgccccatctacaacgctctcgaccccagcgaccagtga

SEQ ID NO: 3 - DNA, 10263 bp, Artifical Sequence
  Synthetic construct
aaaataatcaattaggggttccgcgcaacaatttccccgaaaaaagtgcccacctaaattgtaaagcgtttaatatttttgtttaaaattcgcgttaaattttttgttaaatcagctcatttttttaaccaataggccgaaaatcggcaaaaatcccttataaatcaaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtacgtaccgggcccctagtatgtatgtaagttaataaaacccatttttgcggaaagtagataaaaaaaacattttttttttttactgcactggatatcattgaacttatctgatcagttttaaatttacttcgatccaagggtatttgatgtaccaggttctttcgattacctctcactcaaaatgacattccactcaaagtcagcgctgtttgcctccttctctgtccacagaaatatcgccgtctctttcgccgctgcgtccgctatctctttcgccaccgtttgtagcgttacgtagcgtcaatgtccgccttcagttgcattttgtcagcggtttcgtgacgaagctccaagcggtttacgccatcaattaaacacaaagtgctgtgccaaaactcctctcgcttcttatttttgtttgttttttgagtgattggggtggtgattggttttgggtgggtaagcaggggaaagtgtgaaaaatcccggcaatgggccaagaggatcaggagctattaattcgcggaggcagcaaacacccatctgccgagcatctgaacaatgtgagtagtacatgtgcatacatcttaagttcacttgatctataggaactgcgattgcaacatcaaattgtctgcggcgtgagaactgcgacccacaaaaatcccaaaccgcaattgcacaaacaaatagtgacacgaaacagattattctggtagctgttctcgctatataagacaatttttgagatcatatcatgatcaagacatctaaaggcattcattttcgactatattcttttttacaaaaaatataacaaccagatattttaagctgatcctagatgcacaaaaaataaataaaagtataaacctacttcgtaggatacttcggggtactttttgttcggggttagatgagcataacgcttgtagttgatatttgagatcccctatcattgcagggtgacagcggagcggcttcgcagagctgcattaaccagggcttcgggcaggccaaaaactacggcacgctccggccacccagtccgccggaggactccggttcagggagcggccaactagccgagaacctcacctatgcctggcacaatatggacatctttggggcggtcaatcagccgggctccggatggcggcagctggtcaaccggacacgcggactattctgcaacgagcgacacataccggcgcccaggaaacatttgctcaagaacggtgagtttctattcgcagtcggctgatctgtgtgaaatcttaataaagggtccaattaccaatttgaaactcagtttgcggcgtggcctatccgggcgaacttttggccgtgatgggcagttccggtgccggaaagacgaccctgctgaatgcccttgcctttcgatcgccgcagggcatccaagtatcgccatccgggatgcgactgctcaatggccaacctgtggacgccaaggagatgcaggccaggtgcgcctatgtccagcaggatgacctctttatcggctccctaacggccagggaacacctgattttccaagccatggtgcggatgccacgacatctgacctatcggcagcgagtggcccgcgtggatcaggtgatccaggagctttcgctcagcaaatgtcagcacacgatcatcggtgtgcccggcagggtgaaaggtctgtccggcggagaaaggaagcgtctggcattcgcctccgaggctctaaccgatccgccgcttctgatctgcgatgagcccacctccggactggactcctttaccgcccacagcgtcgtccaggtgctgaagaagctgtcgcagaagggcaagaccgtcatcctgaccattcatcagccgtcttccgagctgtttgagctctttgacaagatccttctgatggccgagggcagggtagctttcttgggcactcccagcgaagccgtcgacttcttttcctagtgagttcgatgtgtttattaagggtatctagtattacataacatctcaactcctatccagcgtgggtgcccagtgtcctaccaactacaatccggcggacttttacgtacaggtgttggccgttgtgcccggacgggagatcgagtcccgtgatcggatcgccaagatatgcgacaattttgccattagcaaagtagcccgggatatggagcagttgttggccaccaaaaatctggagaagccactggagcagccggagaatgggtacacctacaaggccacctggttcatgcagttccgggcggtcctgtggcgatcctggctgtcggtgctcaaggaaccactcctcgtaaaagtgcgacttattcagacaacggtgagtggttccagtggaaacaaatgatataacgcttacaattcttggaaacaaattcgctagattttagatagaattgcctgattccacacccttcttagtttttttcaatgagatgtatagtttatagttttgcagaagataaataaatttcatttaactcgcgaatattaatgagatgcgagtaacattttaatttgcagatggttgccatcttgattggcctcatctttttgggccaacaactcacgcaagtgggtgtgatgaatatcaacggagccatcttcctcttcctgaccaacatgacctttcaaaacgtctttgccacgataaatgtaagtcatgtttagaatacatttgcatttcaataatttactaactttctaatgaatcgattcgatttaggtgttcacctcagagctgccagtttttatgagggaggcccgaagtcgactttatcgctgtgacacatactttctgggcaaaacgattgccgaattgccgctttttctcacagtgccactggtcttcacggcgattgcctatccgatgatcggactgcgggccggagtgctgcacttcttcaactgcctggcgctggtcactctggtggccaatgtgtcaacgtccttcggatatctaatatcctgcgccagctcctcgacctcgatggcgctgtctgtgggtccgccggttatcataccattcctgctctttggcggcttcttcttgaactcgggctcggtgccagtatacctcaaatggttgtcgtacctctcatggttccgttacgccaacgagggtctgctgattaaccaatgggcggacgtggagccgggcgaaattagctgcacatcgtcgaacaccacgtgccccagttcgggcaaggtcatcctggagacgcttaacttctccgccgccgatctgccgctggactacgtgggtctggccattctcatcgtgagcttccgggtgctcgcatatctggctctaagacttcgggcccgacgcaaggagtagccgacatatatccgaaataactgcttgttttttttttttaccattattaccatcgtgtttactgtttattgccccctcaaaaagctaatgtaattatatttgtgccaataaaaacaagatatgacctatagaatacaagtatttccccttcgaacatccccacaagtagactttggatttgtcttctaaccaaaagacttacacacctgcataccttacatcaaaaactcgtttatcgctacataaaacaccgggatatattttttatatacatacttttcaaatcgcgcgccctcttcataattcacctccaccacaccacgtttcgtagttgctctttcgctgtctcccacccgctctccgcaacacattcaccttttgttcgacgaccttggagcgactgtcgttagttccgcgcgattcggttcgctcaaatggttccgagtggttcatttcgtctcaatagaaattagtaataaatatttgtatgtacaatttatttgctccaatatatttgtatatatttccctcacagctatatttattctaatttaatattatgactttttaaggtaattttttgtgacctgttcggagtgattagcgttacaatttgaactgaaagtgacatccagtgtttgttccttgtgtagatgcatctcaaaaaaatggtgggcataatagtgttgtttatatatatcaaaaataacaactataataataagaatacatttaatttagaaaatgcttggatttcactggaactaggctagcataacttcgtataatgtatgctatacgaagttatgctagcggatccgggaattgggaattcacgtaagtactgtctgcagcgtaagcttcgtacgtagcgaccgtctcaaagtactgcctttctgcgttggaaaacatcgcctttttcgtccaaaaggagtccccaggttcgatccgcatggcgttgtgcgtgcgtgcctttcttttcaaatgattacggctattaacttgggggcgttaagttggaaacacgtaaattgcagactgcgattagagtgaccatgagtaggagttcaaaatctcctgacatcattttcttaaaacctgctttgttttttacatttctatttaatataactcctatttgaataaaaaaacaaaacaagtttagatgttaagatattaactacatcctttgctccaaagggagaggggaagttatggagttaattaatttgctgttggaaatcaatatggagtcagaaatataatgatttactaaaccttattgaatcggtaacgatgcgaatttatattaaaatagcttttatgaaacattcaacaaaaatattattaatgttggcccactttagcaaccggttaggtctaccggttgggcaagcaaagattcacgccctggttcgagtcccaactagtcctgcaaaataccgcagcaagttttagagagaccaagtgccattacctctcccacttcagttatcggttatgcggcgtttaagtcgacagcttgccgtctctagctccggtgcctatataaagcagcccgctttccacatttcatattcgttttacgtttgtcaagcctcatagccggcagttcgaacgtatacgctctctgagtcagacctcgaaatcgtagctctacacaattctgtgaattttccttgtcgcgtgtgaaacacttccaaggcaatcttacaaaatgaagtttgttattgttctggcttgcctgttggcagttgtctttgccgctattggtccagttgcagatctcactatcagcaacggagcggttagtcctgatggattctcccgccaagccatattggtgaacgatgtgttcccgtcgccgttgataacgggaaataaaggtgacaggtttcagctcaacgtaatagacaacatgacaaatcacacgatgctcaaatccacaagtatccattggcatggttttttccaacatggcacaaattgggctgatggccctgcattcgtcaaccaatgtcccattagtacgggccacgcttttctgtatgattttcaggttccggatcaggcgggtaccttctggtatcactcgcatttgagcactcagtactgcgacggcctgcgtggtccaatcgtagtctatgatccccaggacccgcataaaagtctgtatgatgttgatgatgacagtacagtgataaccctcgccgactggtatcatttggcagctaaggtcggctcgccggtaccaacagccgacgcaacactcataaatggcctgggtaggtccatagacaccctgaacgcagatttggcggttattaccgttacaaaaggcaagaggtatcgcttccgtttggtcagtttgagttgcgacccaaatcatgttttctccattgatggtcattcgttgaccgtaattgaggcggattccgtaaacttgaagcctcaaacggtagatagtatccaaatctttgcggcccaacggtatagcttcgtcctgaatgctgatcaagatgtgggcaactactggattcgagctctgccgaatagtggcacacgcaactttgacggaggtgttaattcggcgatcctccgctatgatggcgccgcgcccgtcgaaccgacaaccagtcaaacgcccagcacgaatccactcgtagagtcggccctgacaacattggagggaacagcagcgccgggtagccctgctccgggaggtgtagacctcgctctgaacatggcgtttggctttgctggcggcaagtttaccataaatggagcaagctttactcccccgaccgtccctgtgctgttgcagatactctccggtgcgcagtcggcccaagatctgctcccgtcgggctccgtttattccctgcccgccaatgcagacatcgaaatcagcttgccagcaacggctgccgctcccggtttcccacacccgtttcatctccacggtcatacttttgctgtggttcgcagcgcaggctccagtacatacaattatgagaatcccgtctaccgtgacgtcgtttcgactggctcgcccggcgataatgtcaccatccgtttccgaacggataacccgggcccatggtttctgcattgtcatatagactttcatttggaggcgggttttgcagtggttatggccgaggatatcccggaggttgccgccactaaccccgtaccacaagcatggtcggatctgtgccccacatacgacgcgctgtcgccggacgatcagtaagcccgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgcccaaactcatcaatgtatcttagcggctcgagggtacctctagagatccactagtgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacactagttctagccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttcctccgaaaagtgccacctaaattgtaagcgttaatatttttgttaaaattcgcgttaaattttttgttaaatcagctcatttttttaaccaataggccgaaatcggcaaaaatccccttataaatcaaaaagaatagaccgagatagggttgagtgttgttcccagttttggc

SEQ ID NO: 4 - DNA, 10272 bp, Artificial Sequence
  Synthetic construct
aaaataatcaattaggggttccgcgcaacaatttccccgaaaaaagtgcccacctaaattgtaaagcgtttaatatttttgtttaaaattcgcgttaaattttttgttaaatcagctcatttttttaaccaataggccgaaaatcggcaaaaatcccttataaatcaaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtacgtaccgggcccctagtatgtatgtaagttaataaaacccatttttgcggaaagtagataaaaaaaacattttttttttttactgcactggatatcattgaacttatctgatcagttttaaatttacttcgatccaagggtatttgatgtaccaggttctttcgattacctctcactcaaaatgacattccactcaaagtcagcgctgtttgcctccttctctgtccacagaaatatcgccgtctctttcgccgctgcgtccgctatctctttcgccaccgtttgtagcgttacgtagcgtcaatgtccgccttcagttgcattttgtcagcggtttcgtgacgaagctccaagcggtttacgccatcaattaaacacaaagtgctgtgccaaaactcctctcgcttcttatttttgtttgttttttgagtgattggggtggtgattggttttgggtgggtaagcaggggaaagtgtgaaaaatcccggcaatgggccaagaggatcaggagctattaattcgcggaggcagcaaacacccatctgccgagcatctgaacaatgtgagtagtacatgtgcatacatcttaagttcacttgatctataggaactgcgattgcaacatcaaattgtctgcggcgtgagaactgcgacccacaaaaatcccaaaccgcaattgcacaaacaaatagtgacacgaaacagattattctggtagctgttctcgctatataagacaatttttgagatcatatcatgatcaagacatctaaaggcattcattttcgactatattcttttttacaaaaaatataacaaccagatattttaagctgatcctagatgcacaaaaaataaataaaagtataaacctacttcgtaggatacttcggggtactttttgttcggggttagatgagcataacgcttgtagttgatatttgagatcccctatcattgcagggtgacagcggagcggcttcgcagagctgcattaaccagggcttcgggcaggccaaaaactacggcacgctccggccacccagtccgccggaggactccggttcagggagcggccaactagccgagaacctcacctatgcctggcacaatatggacatctttggggcggtcaatcagccgggctccggatggcggcagctggtcaaccggacacgcggactattctgcaacgagcgacacataccggcgcccaggaaacatttgctcaagaacggtgagtttctattcgcagtcggctgatctgtgtgaaatcttaataaagggtccaattaccaatttgaaactcagtttgcggcgtggcctatccgggcgaacttttggccgtgatgggcagttccggtgccggaaagacgaccctgctgaatgcccttgcctttcgatcgccgcagggcatccaagtatcgccatccgggatgcgactgctcaatggccaacctgtggacgccaaggagatgcaggccaggtgcgcctatgtccagcaggatgacctctttatcggctccctaacggccagggaacacctgattttccaagccatggtgcggatgccacgacatctgacctatcggcagcgagtggcccgcgtggatcaggtgatccaggagctttcgctcagcaaatgtcagcacacgatcatcggtgtgcccggcagggtgaaaggtctgtccggcggagaaaggaagcgtctggcattcgcctccgaggctctaaccgatccgccgcttctgatctgcgatgagcccacctccggactggactcctttaccgcccacagcgtcgtccaggtgctgaagaagctgtcgcagaagggcaagaccgtcatcctgaccattcatcagccgtcttccgagctgtttgagctctttgacaagatccttctgatggccgagggcagggtagctttcttgggcactcccagcgaagccgtcgacttcttttcctagtgagttcgatgtgtttattaagggtatctagtattacataacatctcaactcctatccagcgtgggtgcccagtgtcctaccaactacaatccggcggacttttacgtacaggtgttggccgttgtgcccggacgggagatcgagtcccgtgatcggatcgccaagatatgcgacaattttgccattagcaaagtagcccgggatatggagcagttgttggccaccaaaaatctggagaagccactggagcagccggagaatgggtacacctacaaggccacctggttcatgcagttccgggcggtcctgtggcgatcctggctgtcggtgctcaaggaaccactcctcgtaaaagtgcgacttattcagacaacggtgagtggttccagtggaaacaaatgatataacgcttacaattcttggaaacaaattcgctagattttagatagaattgcctgattccacacccttcttagtttttttcaatgagatgtatagtttatagttttgcagaagataaataaatttcatttaactcgcgaatattaatgagatgcgagtaacattttaatttgcagatggttgccatcttgattggcctcatctttttgggccaacaactcacgcaagtgggtgtgatgaatatcaacggagccatcttcctcttcctgaccaacatgacctttcaaaacgtctttgccacgataaatgtaagtcatgtttagaatacatttgcatttcaataatttactaactttctaatgaatcgattcgatttaggtgttcacctcagagctgccagtttttatgagggaggcccgaagtcgactttatcgctgtgacacatactttctgggcaaaacgattgccgaattgccgctttttctcacagtgccactggtcttcacggcgattgcctatccgatgatcggactgcgggccggagtgctgcacttcttcaactgcctggcgctggtcactctggtggccaatgtgtcaacgtccttcggatatctaatatcctgcgccagctcctcgacctcgatggcgctgtctgtgggtccgccggttatcataccattcctgctctttggcggcttcttcttgaactcgggctcggtgccagtatacctcaaatggttgtcgtacctctcatggttccgttacgccaacgagggtctgctgattaaccaatgggcggacgtggagccgggcgaaattagctgcacatcgtcgaacaccacgtgccccagttcgggcaaggtcatcctggagacgcttaacttctccgccgccgatctgccgctggactacgtgggtctggccattctcatcgtgagcttccgggtgctcgcatatctggctctaagacttcgggcccgacgcaaggagtagccgacatatatccgaaataactgcttgttttttttttttaccattattaccatcgtgtttactgtttattgccccctcaaaaagctaatgtaattatatttgtgccaataaaaacaagatatgacctatagaatacaagtatttccccttcgaacatccccacaagtagactttggatttgtcttctaaccaaaagacttacacacctgcataccttacatcaaaaactcgtttatcgctacataaaacaccgggatatattttttatatacatacttttcaaatcgcgcgccctcttcataattcacctccaccacaccacgtttcgtagttgctctttcgctgtctcccacccgctctccgcaacacattcaccttttgttcgacgaccttggagcgactgtcgttagttccgcgcgattcggttcgctcaaatggttccgagtggttcatttcgtctcaatagaaattagtaataaatatttgtatgtacaatttatttgctccaatatatttgtatatatttccctcacagctatatttattctaatttaatattatgactttttaaggtaattttttgtgacctgttcggagtgattagcgttacaatttgaactgaaagtgacatccagtgtttgttccttgtgtagatgcatctcaaaaaaatggtgggcataatagtgttgtttatatatatcaaaaataacaactataataataagaatacatttaatttagaaaatgcttggatttcactggaactaggctagcataacttcgtataatgtatgctatacgaagttatgctagcggatccgggaattgggaattcacgtaagtactgtctgcagcgtaagcttcgtacgtagcgaccgtctcaaagtactgcctttctgcgttggaaaacatcgcctttttcgtccaaaaggagtccccaggttcgatccgcatggcgttgtgcgtgcgtgcctttcttttcaaatgattacggctattaacttgggggcgttaagttggaaacacgtaaattgcagactgcgattagagtgaccatgagtaggagttcaaaatctcctgacatcattttcttaaaacctgctttgttttttacatttctatttaatataactcctatttgaataaaaaaacaaaacaagtttagatgttaagatattaactacatcctttgctccaaagggagaggggaagttatggagttaattaatttgctgttggaaatcaatatggagtcagaaatataatgatttactaaaccttattgaatcggtaacgatgcgaatttatattaaaatagcttttatgaaacattcaacaaaaatattattaatgttggcccactttagcaaccggttaggtctaccggttgggcaagcaaagattcacgccctggttcgagtcccaactagtcctgcaaaataccgcagcaagttttagagagaccaagtgccattacctctcccacttcagttatcggttatgcggcgtttaagtcgacagcttgccgtctctagctccggtgcctatataaagcagcccgctttccacatttcatattcgttttacgtttgtcaagcctcatagccggcagttcgaacgtatacgctctctgagtcagacctcgaaatcgtagctctacacaattctgtgaattttccttgtcgcgtgtgaaacacttccaaggcaatcttacaaaatgaagtttgttattgttctggcttgcctgttggcagttgtctttgccgctattggccctgttaccgacttgacggttacggatgccaacatcagtcccgacggatttacgcgcgccggtatagtggtcaacaacgtgttccccgctcccctgattacgggacaaaaaggcgacaggtttcagctgaatctggtcaaccaaatgagtaatcatacgatgttgaagacaacttcgatacactggcacggtttttttcaaaagggaactaattgggcggatggacccgcattcgtgaatcagtgtcctatcgctagcggtaattcgtttctgtatgactttcaggtgcccgatcaagctggaactttctggtaccactcccacctgtccacgcagtattgtgacggtctccgcggacctttcgtagtttatgacccaactgaccctcacctgtcgctgtatgacgtggatgacgatagcacagttataacgctggcggattggtaccatgtggccgcacgcctgggaccgcgcttcccgttgggtgctgactccactctgatcaacggtttgggcaggtccaccgcgactccgactgctgatctggctgtgatcagtgtaaccaaaggtaaacgttaccggtttaggctggtgagtatttcctgcgatccgaaccataccttcagcattgacggtcacaagctcaccgtcatcgaagctgatggcatttccactcaacccgtcactggcattgactccattcaaatttttgccgcacagaggtacagcttcgtcctcaccgccgaccaggatgtagacaattactgggttagggctaaccctaattttggtactactggatttgccggaggcattaacagcgcgattctccgttatgatggtgcacctgcggttgaacccactacctcccaaaccggtacgaatctgctcgtggagacagatctgcatcctctgagcacaatgcccgttccgggactgccgacacaaggtggtgccgattttaatctcaatctcgcctttaactttaacggttcggacttctttattaacggtgcttcgttcgttccaccgaccgtccctgtcctcctccagataatcagcggtgcgaacagcgcccaagatttgctcccaagtggttcggtatatgcactcccctcgaatagcagcatcgaactcacattcccggccactgctgcagcgccaggcgccccccatccattccacttgcatggccacgcttttgccgtagtacgcagtgctggaagtactgtttataactacgacaacccggtattcagggatgtagtttccactggtacgcccgctgcgggcgacaacgtcaccatccgattccaaacggacaatcctggaccatggtttttgcattgtcacatcgacttccacctcgatgctggcttcgctgtcgtgttcgcggaagacctcccggacgttgtaagtgcaaaccccgtgccccaagcgtggagcgatttgtgccccatctataacgctctcgatccgtcggatcagtaagcccgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgcccaaactcatcaatgtatcttagcggctcgagggtacctctagagatccactagtgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacactagttctagccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttcctccgaaaagtgccacctaaattgtaagcgttaatatttttgttaaaattcgcgttaaattttttgttaaatcagctcatttttttaaccaataggccgaaatcggcaaaaatccccttataaatcaaaaagaatagaccgagatagggttgagtgttgttcccagttttggc

SEQ ID NO: 5 - DNA, 22 bp, Artificial Sequence
  Sequencing primer
gcttggatttcactggaactag

SEQ ID NO: 6 - DNA, 20 bp, Artificial Sequence
  Sequning primer
tcgagaccgtgacctacatc

SEQ ID NO: 7 - DNA, 20 bp, Artificial Sequence
  Sequencing primer
cgtttgtcaagcctcatagc

SEQ ID NO: 8 - DNA, 25 bp, Artificial Sequence
  Sequencing primer
ttcattttatgtttcaggttcaggg

SEQ ID NO: 9 - DNA, 20 bp, Artificial Sequence
  Sequencing primer
tgactttcaggtgcccgatc

SEQ ID NO: 10 - DNA, 21 bp, Artificial Sequence
  Sequencing primer
gccgggaatgtgagttcgatg

SEQ ID NO: 11 - DNA, 1118 bp, Artificial Sequence
  gBlock
agcgtaagcttcgtacgtagcgaccgtctcaaagtactgcctttctgcgttggaaaacatcgcctttttcgtccaaaaggagtccccaggttcgatccgcatggcgttgtgcgtgcgtgcctttcttttcaaatgattacggctattaacttgggggcgttaagttggaaacacgtaaattgcagactgcgattagagtgaccatgagtaggagttcaaaatctcctgacatcattttcttaaaacctgctttgttttttacatttctatttaatataactcctatttgaataaaaaaacaaaacaagtttagatgttaagatattaactacatcctttgctccaaagggagaggggaagttatggagttaattaatttgctgttggaaatcaatatggagtcagaaatataatgatttactaaaccttattgaatcggtaacgatgcgaatttatattaaaatagcttttatgaaacattcaacaaaaatattattaatgttggcccactttagcaaccggttaggtctaccggttgggcaagcaaagattcacgccctggttcgagtcccaactagtcctgcaaaataccgcagcaagttttagagagaccaagtgccattacctctcccacttcagttatcggttatgcggcgtttaagtcgacagcttgccgtctctagctccggtgcctatataaagcagcccgctttccacatttcatattcgttttacgtttgtcaagcctcatagccggcagttcgaacgtatacgctctctgagtcagacctcgaaatcgtagctctacacaattctgtgaattttccttgtcgcgtgtgaaacacttccaaggcggccgcccgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgcccaaactcatcaatgtatcttagcggctcgagggtacctctag

SEQ ID NO: 12 - DNA, 1600 bp, Artificial Sequence
  gBlock
gtgtgaaacacttccaaggcaatcttacaaaatgaagtttgttattgttctggcttgcctgttggcagttgtctttgccgctattggccctgttaccgacttgacggttacggatgccaacatcagtcccgacggatttacgcgcgccggtatagtggtcaacaacgtgttccccgctcccctgattacgggacaaaaaggcgacaggtttcagctgaatctggtcaaccaaatgagtaatcatacgatgttgaagacaacttcgatacactggcacggtttttttcaaaagggaactaattgggcggatggacccgcattcgtgaatcagtgtcctatcgctagcggtaattcgtttctgtatgactttcaggtgcccgatcaagctggaactttctggtaccactcccacctgtccacgcagtattgtgacggtctccgcggacctttcgtagtttatgacccaactgaccctcacctgtcgctgtatgacgtggatgacgatagcacagttataacgctggcggattggtaccatgtggccgcacgcctgggaccgcgcttcccgttgggtgctgactccactctgatcaacggtttgggcaggtccaccgcgactccgactgctgatctggctgtgatcagtgtaaccaaaggtaaacgttaccggtttaggctggtgagtatttcctgcgatccgaaccataccttcagcattgacggtcacaagctcaccgtcatcgaagctgatggcatttccactcaacccgtcactggcattgactccattcaaatttttgccgcacagaggtacagcttcgtcctcaccgccgaccaggatgtagacaattactgggttagggctaaccctaattttggtactactggatttgccggaggcattaacagcgcgattctccgttatgatggtgcacctgcggttgaacccactacctcccaaaccggtacgaatctgctcgtggagacagatctgcatcctctgagcacaatgcccgttccgggactgccgacacaaggtggtgccgattttaatctcaatctcgcctttaactttaacggttcggacttctttattaacggtgcttcgttcgttccaccgaccgtccctgtcctcctccagataatcagcggtgcgaacagcgcccaagatttgctcccaagtggttcggtatatgcactcccctcgaatagcagcatcgaactcacattcccggccactgctgcagcgccaggcgccccccatccattccacttgcatggccacgcttttgccgtagtacgcagtgctggaagtactgtttataactacgacaacccggtattcagggatgtagtttccactggtacgcccgctgcgggcgacaacgtcaccatccgattccaaacggacaatcctggaccatggtttttgcattgtcacatcgacttccacctcgatgctggcttcgctgtcgtgttcgcggaagacctcccggacgttgtaagtgcaaaccccgtgccccaagcgtggagcgatttgtgccccatctataacgctctcgatccgtcggatcagtaagcccgactctagatcataatc

SEQ ID NO: 13 - DNA, 1591 bp, Artificial Sequence
  gBlock
gtgtgaaacacttccaaggcaatcttacaaaatgaagtttgttattgttctggcttgcctgttggcagttgtctttgccgctattggtccagttgcagatctcactatcagcaacggagcggttagtcctgatggattctcccgccaagccatattggtgaacgatgtgttcccgtcgccgttgataacgggaaataaaggtgacaggtttcagctcaacgtaatagacaacatgacaaatcacacgatgctcaaatccacaagtatccattggcatggttttttccaacatggcacaaattgggctgatggccctgcattcgtcaaccaatgtcccattagtacgggccacgcttttctgtatgattttcaggttccggatcaggcgggtaccttctggtatcactcgcatttgagcactcagtactgcgacggcctgcgtggtccaatcgtagtctatgatccccaggacccgcataaaagtctgtatgatgttgatgatgacagtacagtgataaccctcgccgactggtatcatttggcagctaaggtcggctcgccggtaccaacagccgacgcaacactcataaatggcctgggtaggtccatagacaccctgaacgcagatttggcggttattaccgttacaaaaggcaagaggtatcgcttccgtttggtcagtttgagttgcgacccaaatcatgttttctccattgatggtcattcgttgaccgtaattgaggcggattccgtaaacttgaagcctcaaacggtagatagtatccaaatctttgcggcccaacggtatagcttcgtcctgaatgctgatcaagatgtgggcaactactggattcgagctctgccgaatagtggcacacgcaactttgacggaggtgttaattcggcgatcctccgctatgatggcgccgcgcccgtcgaaccgacaaccagtcaaacgcccagcacgaatccactcgtagagtcggccctgacaacattggagggaacagcagcgccgggtagccctgctccgggaggtgtagacctcgctctgaacatggcgtttggctttgctggcggcaagtttaccataaatggagcaagctttactcccccgaccgtccctgtgctgttgcagatactctccggtgcgcagtcggcccaagatctgctcccgtcgggctccgtttattccctgcccgccaatgcagacatcgaaatcagcttgccagcaacggctgccgctcccggtttcccacacccgtttcatctccacggtcatacttttgctgtggttcgcagcgcaggctccagtacatacaattatgagaatcccgtctaccgtgacgtcgtttcgactggctcgcccggcgataatgtcaccatccgtttccgaacggataacccgggcccatggtttctgcattgtcatatagactttcatttggaggcgggttttgcagtggttatggccgaggatatcccggaggttgccgccactaaccccgtaccacaagcatggtcggatctgtgccccacatacgacgcgctgtcgccggacgatcagtaagcccgactctagatcataatc

(19) United States
(12) Patent Application Publication
(10) Pub. No.: US 2024/0099281 A1
(43) Pub. Date: Mar. 28, 2024

(54) TRANSGENIC ANIMALS AND PREPARATIONS THEREOF FOR BIOREMEDIATION

(71) Applicant: MACQUARIE UNIVERSITY, North Ryde (AU)

(72) Inventors: Maciej MASELKO, North Ryde (AU); Kaitlyn TEPPER, North Ryde (AU); Michael CLARK, North Ryde (AU)

(21) Appl. No.: 18/257,767

(22) PCT Filed: Dec. 13, 2021
(86) PCT No.: PCT/AU2021/051483

§ 371 Date: Jun. 15, 2023

(30) Foreign Application Priority Data
Dec. 15, 2020 (AU) 2020904672

(51) Int. Cl. A01K 67/033; A62D 3/02; C12N 9/02

(52) U.S. Cl. CPC A01K 67/0339; A62D 3/02; C12N 9/0061; C12Y 110/03002; A01K 2227/706; A01K 2267/01; A62D 2101/28

(57) ABSTRACT

There is provided a transgenic insect comprising a heterologous nucleic acid encoding a fungal laccase wherein the heterologous nucleic acid is integrated in the insect genome, wherein the heterologous nucleic acid is operably linked to a promoter, and expression of the laccase allows for production of an active fungal laccase in the insect. The insect or laccase preparations comprising meal or powder made from the insect are used to reduce or remove phenolic contaminants (such as BPA) or aromatic components (such as indigo dyes) from waste.

### TECHNICAL FIELD

The technology relates to transgenic animals such as insects, for example black soldier fly larvae, expressing a fungal laccase and use of the transgenic animals such as insects for processing of laccase substrates. Target substrates include industrial dyes such as indigo carmine and plasticizers such as BPA.

### CROSS REFERENCE TO RELATED APPLICATION

This application claims the benefit of Australian Provisional Application No. 2020904672 filed 15 Dec. 2020, the entire content of which is incorporated by reference herein.

### BACKGROUND

Laccases are oxidoreductive copper-containing enzymes found in microbes, fungi, and higher organisms. Laccase enzymes are used for many applications, including pulp and textile bleaching, treatment of pulp waste water, de-inking, industrial color removal, bleaching laundry detergents, oral care teeth whiteners, and as catalysts or facilitators for polymerization and oxidation reactions.

Laccases have a broad substrate specificity including aminophenols, polyphenols, ortho- and para-diphenols, polyamines and aryl diamines as well as some inorganic ions. Some of these substrates, such as bisphenol A (BPA) and indigo carmine are industrial pollutants that can be harmful to human and environmental health. Laccases can assist in the degradation of these chemicals, however, their production and purification on large scales is costly. Laccases are known to be produced by a wide variety of fungi, including species of the genera Aspergillus, Neurospora, Podospora, Botrytis, Pleurotus, Fomes, Phlebia, Trametes, Polyporus, Stachybotrys, Rhizoctonia, Bipolaris, Curvularia, Amerosporium, and Lentinus.

For many applications, laccase efficiency can be improved through the use of a mediator, also known as an enhancing agent. Systems that include a laccase and a mediator are known in the art as laccase-mediator systems. However, the majority of mediators are expensive, toxic, or both thereby limiting the applicability of laccase-mediator systems.

There are several known mediators for use in a laccase-mediator system. These include HBT (1-hydroxybenzotriazole), ABTS [2,2′-azinobis(3-ethylbenzothiazoline-6-sulfinic acid)], NHA (N-hydroxyacetanilide), NEIAA (N-acetyl-N-phenylhydroxylamine), HBTO (3-hydroxy 1,2,3-benzotriazin-4(3H)-one), and VIO (violuric acid). In addition, there are several compounds containing NH—OH or N—O that have been found to be useful as mediators.

Functional groups and substituents have large effects on mediator efficiency. Even within the same class of compounds, a substituent can change the laccase specificity towards a substrate, thereby increasing or decreasing mediator efficiency greatly. In addition, a mediator may be effective for one particular application but unsuitable for another application. Another drawback for current mediators is their tendency to polymerize during use.

The use of a laccase-mediator systems increases the complexity and cost of using laccases, particularly in the context of waste remediation where a simple, robust and cost-effective products are ideal.

The use of biological agents such as laccase for the purpose of degrading or detoxifying harmful compounds such as indigo dyes and BPA involves the use of free enzymes, or microbes. These are often costly or impractical. Major challenges include lack of activity in the selected environment, accessibility of target compounds, and transgene biocontainment when using live genetically engineered organisms.

Thus, there is a need to for methods and systems to produce laccase preparations, preferably without the need to add a mediator to the preparation.

### SUMMARY

In a first aspect, there is provided a transgenic insect capable of expressing an active laccase, the insect comprising a heterologous nucleic acid encoding the laccase wherein the nucleic acid encoding the laccase is operably linked to a promoter.

The heterologous nucleic acid may comprise SEQ ID NO: 1 or a sequence at least 80%, 85%, 90%, 95%, 97%, or 99% identical to SEQ ID NO: 1. In one embodiment the laccase encoded by the heterologous nucleic acid is at least 80%, 85%, 90%, 95%, 97%, 99% or 100% identical to the laccase encoded by SEQ ID NO: 1.

The fungal laccase may be a T trogii laccase, for example a T trogii laccase encoded by SEQ ID NO: 1, or a T trogii laccase with at least 80% sequence identity to a T trogii laccase encoded by SEQ ID NO: 1.

In some embodiments the heterologous nucleic acid further comprises a sequence encoding a signal peptide in frame with the sequence encoding the fungal laccase.

The signal peptide may be the larval cuticle protein 9 signal peptide, or a sequence at least 80%, 85%, 90%, 95%, 97%, or 99% identical to larval cuticle protein 9.

The promoter may be a truncated tubulin alpha promoter.

In one embodiment the transgenic insect is selected from the genus Hermetia, Drosophila, and Tenebrio.

For example the insect may be Hermetia illucens, Drosophila melanogaster or Tenebrio molitor.

In a second aspect there is provided a laccase preparation comprising the transgenic insect of the first aspect, or a portion thereof. In some embodiments the transgenic insect is fully or partially dried. The transgenic insect may be in the form of a meal or a powder.

The laccase preparation may further comprise one or more of a preservative, anti-caking agent or surfactant.

In some embodiments the laccase preparation does not comprise an added laccase mediator.

In a third aspect there is provided a method for removing or reducing the level of a phenolic or aromatic contaminant in a substance comprising contacting the substance with an effective amount of a laccase preparation of the second aspect.

The method may further comprise incubating the laccase preparation and the substance, preferably at ambient temperature.

In a fourth aspect there is provided a method for removing or reducing the level of a phenolic or aromatic contaminant in a substance comprising contacting the substance with the transgenic insect of the first aspect under conditions suitable for the insect to feed on the substance.

The substance may be liquid waste or wet waste. The phenolic contaminant may be BPA. The aromatic contaminant may be an indigo dye.

The concentration of the phenolic or aromatic contaminant in the substance may be reduced by about 5%, 10%, 15%, 20%, 25%, 30%, 35%, 40%, 45%, 50%, 55%, 56%, 60%, 65%, 70%, 75%, 80%, 85% or at least about 90%.

In one embodiment the phenolic contaminant is BPA.

In another embodiment the aromatic contaminant is an indigo dye.

### Definitions

Throughout this specification, unless the context clearly requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, the term ‘consisting essentially of’ means the inclusion of the stated element(s), integer(s) or step(s), but other element(s), integer(s) or step(s) that do not materially alter or contribute to the working of the invention may also be included.

Throughout this specification, the term ‘consisting of’ means consisting only of.

Any discussion of documents, acts, materials, devices, articles or the like which has been included in the present specification is solely for the purpose of providing a context for the present technology. It is not to be taken as an admission that any or all of these matters form part of the prior art base or were common general knowledge in the field relevant to the present technology as it existed before the priority date of each claim of this specification.

Unless the context requires otherwise or specifically stated to the contrary, integers, steps, or elements of the technology recited herein as singular integers, steps or elements clearly encompass both singular and plural forms of the recited integers, steps or elements.

In the context of the present specification the terms ‘a’ and ‘an’ are used to refer to one or more than one (i.e., at least one) of the grammatical object of the article. By way of example, reference to ‘an element’ means one element, or more than one element.

In the context of the present specification the term ‘about’ means that reference to a figure or value is not to be taken as an absolute figure or value, but includes margins of variation above or below the figure or value in line with what a skilled person would understand according to the art, including within typical margins of error or instrument limitation. In other words, use of the term ‘about’ is understood to refer to a range or approximation that a person or skilled in the art would consider to be equivalent to a recited value in the context of achieving the same function or result.

A ‘promoter’ is defined as an array of nucleic acid control sequences that direct transcription of an operably linked nucleic acid. Promoters include necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription.

The term ‘operably linked’ refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

Those skilled in the art will appreciate that the technology described herein is susceptible to variations and modifications other than those specifically described. It is to be understood that the technology includes all such variations and modifications. For the avoidance of doubt, the technology also includes all of the steps, features, and compounds referred to or indicated in this specification, individually or collectively, and any and all combinations of any two or more of said steps, features and compounds.

In order that the present technology may be more clearly understood, preferred embodiments will be described with reference to the following drawings and examples.

### BRIEF DESCRIPTION OF THE DRAWINGS

FIG. 1: In vitro laccase substrate oxidation in fly lysates. (A) Fly lysate oxidation of 1 mg/mL ABTS for 2 hours. n=2 biologically independent replicates for WT, n=6 for all others. (B) Fly lysate degradation of 25 μg/mL BPA for 21 h. n=3 biologically independent replicates. Dm/Tt.Lcc1=D. melanogaster engineered with T. trogii laccase, Dm/Pb.Lac1=D. melanogaster engineered with P. brumalis laccase, WT=wild-type, NFC=No-Fly control. The statistical analysis was conducted using one-way ANOVA using Dunnett's method compared to WT, where ns=not statistically significant (p>0.05). (C) Overlaid representative extracted ion chromatograms (EIC) (m/z 227.15) displaying the BPA 132 peak for samples shown in (B). n=3 biologically independent replicates.

FIG. 2: In vivo laccase enzyme activity. In vivo degradation of 25 μg/mL BPA without ABTS (A) and with 1 mg/mL ABTS (B). The statistical analysis was conducted using one-way ANOVA using Dunnett's method compared to WT and NFC=No-Fly control, where ns=not statistically significant (p>0.05).

FIG. 3: In vivo BPA degradation conditions comparing Dm/Tt.Lcc1 lysate to NFC. Fly lysate was added to media that contained 25 μg/mL BPA with and without 1 mg/mL ABTS. Dm/Tt.Lcc1 lysate was unable to degrade BPA without ABTS. n=3 biologically independent replicates. The statistical analysis was a one-way ANOVA with Dunnett's method. ns=not significant, Dm/Tt.Lcc1=D. melanogaster engineered with T. trogii laccase, NFC=no fly control.

FIG. 4: In vitro activity of lyophilized Dm/Tt.Lcc1 whole-fly powder on laccase substrates. (A) Decolorization (%) of 100 mg/L indigo carmine at 610 nm in whole lyophilized powder resuspended in water after 48 hours. n=3 biologically independent replicates. (B-D) Time course decolorization (%) of 100 mg/mL indigo carmine in the soluble fraction of lyophilized powder in water (B) or supplemented with 10 mM CuSO₄ (C). Data is shown as mean values±standard deviation. Statistical analysis of (C) was performed at end point in (D). n=3 biologically independent replicates. (E, F) Time course degradation of 12.5 μg/mL BPA in the soluble fraction of lyophilized samples in water supplemented with 10 mM CuSO₄ (E). Data is shown as mean values±standard deviation. Statistical analysis of (E) was performed at end point in (F). n=3 biologically independent replicates. Dm/Tt.Lcc1=D. melanogaster engineered with T. trogii laccase, WT=wild-type, NFC=No-Fly control. The statistical analysis was conducted using one-way ANOVA using Dunnett's method compared to WT, where ns=not statistically significant (p>0.05).

### DESCRIPTION OF EMBODIMENTS

The technology described herein generally relates to transgenic insects expressing a laccase enzyme. The insect is used as the base for a laccase preparation containing the expressed laccase. In some embodiments the larval stage of the insect is used as the base for the laccase preparation. The laccase preparation can be used in the treatment of liquid waste or wet waste.

As used herein, the term ‘liquid waste’ refers to any domestic or industrial waste which is essentially or predominantly a liquid. The term includes liquid waste materials containing solids, such as sewage which are liquid in the sense that the majority of the waste material is liquid with solids dispersed or distributed throughout. Examples of liquid waste include municipal wastewater, domestic wastewater, greywater, blackwater, textile wastewater.

As used herein, the term ‘wet waste’ refers to any domestic or industrial biodegradable waste which is essentially or predominantly a solid or semisolid but contains a significant aqueous component. The term includes liquid waste materials that are predominantly solids such as contaminated wetlands, mud, paper pulp (for example paper pulp for de-inking).

Described herein are organisms including animals and insects (e.g. Drosophila melanogaster) that are or can be genetically engineered to express a functional laccase from the fungus Trametes trogii. Engineered D. melanogaster are capable of oxidizing laccase substrates (ABTS (2,2′-azino-bis(3-ethylbenzothiazoline-6-sulfonic acid)) and BPA (bisphenol A)) in vivo when present in their diet. However, in vivo BPA oxidation was dependent on ABTS to act as a mediator.

Exogenous mediators are not necessary for the oxidation of BPA or indigo carmine when the flies were lyophilized and ground into a fine powder. Transgenic organisms can therefore serve as production platforms for T. trogii laccase. The transgenic organisms can be used as the basis for a laccase preparation being a lyophilized powder that can be produced without the need for costly purification or addition of mediator compounds. Transgenic Hermetia illucens (black soldier flies) can be used to add value to organic waste-streams by producing high value laccase powder may be used for bioremediation applications.

### Laccase

In the context of this disclosure, laccases refers to any enzyme comprised by the enzyme classification EC 1.10.3.2-diphenol:oxygen oxidoreductase. Laccase are copper-containing oxidases that utilise molecular oxygen as oxidant and also oxidise phenolic rings to phenoxy radicals. That is, laccases are phenoloxidases capable of oxidizing phenolic and aromatic compounds. Laccases are known from microbial and plant origin. It is contemplated that any laccase known in the art may be used in the recombinant insects described herein and a skilled person will be able to identify suitable laccases.

In some embodiments the laccase is a microbial laccase. The microbial laccase enzyme may be derived from bacteria or fungi (including filamentous fungi and yeasts) and suitable examples include a laccase derivable from a strain of Aspergillus, Neurospora, e.g. N. crassa. Podospora, Botrytis, Collybia, Cerrena, Stachybotrys, Panus, e.g., Panus rudis, Theilava, Fomes, Lentinus, Pleurotus, Tramatella, e.g. T. atroviride, T. trogii, T. villosa and T. versicolor, T. gallica, T. cervine, T. optimum, T. villosa, Antroida, Rhizoctonia, e.g. R. solani, Coprinus, e.g. C. plicatilis and C. cinereus, Psatyrella, Myceliophthora, e.g. M. thermonhila, Schytalidium, Phlebia, e.g. P. radita, or Coriolus, e.g. C. hirsutus, Spongipellis sp., Polyporus, Ceriporiopsis subvermispora, Ganoderma tsunodae, Trichoderma, Pycnoporus sanguineus, Myceliophthora thermophila, and Laccaria bicolor

The laccase may contain various sequence changes from the wild-type or naturally occurring laccase. In this context, the term “% identity” refers to the level of nucleic acid or amino acid sequence identity between the modified laccase and the wild-type laccase. For example, modified laccase may have 80%, 81%, 82%, 83%, 84%, 85%, 86%, 87%, 88%, 89%, 90%, 91%, 92%, 93%, 94%, 95%, 96%, 97%, 98% or 99% identity to its wild-type counterpart, for example SEQ ID NO: 1. In some embodiments the modifications do not alter the enzymatic activity or specificity of the laccase.

In one embodiment the laccase is from a strain of Tramatella, preferably T. troigii. In one embodiment the laccase is a T. trogii laccase, for example a T. trogii laccase having a coding sequence of SEQ ID NO: 1 or a sequence at least 80%, at least 85%, at least 90%, at least 95%, at least 97%, or at least 99% identical to SEQ ID NO: 1.

In some embodiments, it may be desirable to modify the laccase. One of skill will recognize many ways of generating alterations in a given nucleic acid construct. Such well-known methods include site-directed mutagenesis, PCR amplification using degenerate oligonucleotides, exposure of cells containing the nucleic acid to mutagenic agents or radiation, chemical synthesis of a desired oligonucleotide (e.g., in conjunction with ligation and/or cloning to generate a nucleic acid encoding a modified laccase).

In one embodiment the modification involves replacing the portion of the nucleic acid that encodes the endogenous signal peptide and replacing it with a nucleic acid sequence for the insect species of interest such as a Drosophila or Hermetia signal peptide.

In some embodiments the nucleic acid is codon optimized the nucleic sequence for the insect species of interest such as a Drosophila or Hermetia.

In one embodiment positions 1-66 of the SEQ ID NO: 1 (the T. trogii laccase coding region) was replaced with a nucleic acid sequence coding for a Drosophila signal peptide. For example, plasmin pMC-1-2-3 (SEQ ID NO: 3) comprises a sequence encoding the Drosophila larval cuticle protein 9 signal peptide from position 5795-5844 and the T. trogii laccase from 5845-7332.

In some embodiments the nucleic acids encoding the laccase may be conservatively modified. With respect to nucleic acid sequences, conservatively modified refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are ‘silent variations’ which are one species of conservatively modified variations. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a ‘conservatively modification’ where the alteration results in the substitution of an amino acid with a chemically similar amino acid. Conservative substitution tables providing functionally similar amino acids are well known in the art.

The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (N).

A skilled person will recognise that other modifications can be made to the laccase polypeptides or nucleic acids without diminishing their biological activity. Some modifications may be made to facilitate the cloning, expression, and the like. Such modifications are well known to those of skill in the art and include, for example, a methionine added at the amino terminus to provide an initiation site, or additional amino acids that form an epitope tag (e.g., poly His) placed on either terminus to facilitate purification or identification.

In some embodiments, laccase thermostability may be improved. For example, a quadruple mutant L386W/G417L/G57F/K317N of the CotA-laccase from B. pumilus W3, enhances thermal stability and improves dye degradation. Corresponding mutations may be made to the laccase described herein.

In other embodiments, the optimum pH for enzyme activity may be modified. For a laccase from Pycnoporus cinnabarinus having five mutations in the mature protein (N208S, R280H, N331D, D341N, and P394H) has an altered pH optimum. Compared with the parent enzyme at pH 5, the variant showed a similar or higher catalytic efficiency (kcat/KM) towards model substrates: an 18-fold increase for ABTS (nonphenolic substrate), a 5.7-fold increase for sinapic acid (phenolic substrate), and a 1.6-fold increase for 2,6-dimethoxyphenol (DMP). In addition, the optimum pH shifted from 2 to 4.5 for ABTS, and from 3 to 4.5 for DMP. Corresponding mutations may be made to the laccase described herein.

The thermostable laccase mutant MtLT2 from the ascomycete fungus Myceliophthora thermophila contains one conservative mutation (D530E) away from the active site and another mutation (N1095) in the vicinity of the T2/T3 site. The optimum pH of the resulting variant shifted from 4 to 6.5 and catalytic efficiency of the variant increased 31-fold for ABTS and a ninefold for DMP. Corresponding mutations may be made to the laccase described herein

One property particularly relevant for applications of laccase is total turnover (TTN). Total turnover is defined as the amount of converted substrate or generated product per amount of enzyme consumed in the reaction. Typically, laccases become inactivate after a certain number of catalytic cycles or turnovers. Thus, TTN may be interpreted as yield, reflecting the activity and stability of the catalyst during operation, and it is independent of time. In the case of free-radical generating reactions such as phenol oxidation, laccases may be inactivated by oxidation of relevant residues. In C. gallica laccase each of the single mutants F357L and F413A showed similar kinetic constants with syringaldazine, but higher TTN (2- to 2.6-fold) during 4-methoxyphenol oxidation a reaction that generates phenoxy free radicals, indicating that these mutations may increase TTN. Corresponding mutations may be made to the laccase described herein.

Manipulating the properties of the laccase enzymes described herein can be achieved using modern protein engineering tools known in the art. Properties such as optimum pH, organic, thermotolerance, and operational stability may be targeted to modify the laccase.

In order to express a laccase in an insect, a nucleic acid encoding the laccase is incorporated into an expression cassette or expression vector. A typical expression cassette, which may be part of a larger nucleic acid construct such as an expression vector, contains a promoter operably linked to a nucleic acid encoding the laccase and optionally other sequences such as a transcription terminator and a sequence encoding a signal peptide.

The promoter can be constitutive or inducible. Inducible promoters can be advantageous because the insect's cells can be grown to high densities before expression of the laccase polypeptide is induced.

Examples of suitable constitutive promoters are actin 5C promoter, 5′LTR of a COPIA element or the short tubulin alpha promoter.

In one embodiment the short tubulin alpha promoter is used.

As the shortened alpha tubulin promoter is effective it is apparent that a strong ubiquitous promoter that drives the overexpression of enzymes in all tissues can also be used.

Suitable inducible promoters include the metallothionein, HSp-70 promoter, and tetracycline inducible promoters.

The transgenic insects exemplified herein utilise the shortened alpha tubulin promoter to drive midlevel expression across all tissues. This promoter was selected to avoid potential toxicity arising from overexpression and to increase the chances that the enzyme will encounter favourable physiological conditions for activity.

In some embodiments, the laccase is secreted. Laccases for secretion may fuse directly via a linker sequence to a signal peptide, for example at the N-terminus that directs the protein into the endoplasmic reticulum, which is the first step in the pathway leading to secretion from the cell. The signal peptide may be fused to the laccase or the nucleic acid sequence encoding the laccase signal sequence may be replaced by a nucleic acid sequence encoding a heterologous signal peptide.

Numerous signal peptides are known in the art and include signal peptides from multiple sources such as signal peptides from human beta-interferon, human placental alkaline phosphatase, D. melanogaster cuticle protein II, or larval cuticle protein 9, B. mori bombyxin, H. virescens juvenile hormone esterase, T. ni acidic juvenile hormone-suppressible hemolymph protein, flesh fly sarcotoxin IA, honeybee melittin, M. sexta adipokinetic hormone, Lucilia cuprina (Australian sheep blowfly) chymotrypsin, AcMNPV GP67, AcMNPV EGT, Spodoptera litura NPV EFP (envelope fusion protein), and the venom neurotoxin signal peptides from the mite toxin encoded by tox21A and neurotoxins AaIT, LqhIT2, and BjIT (from the scorpion Hottentota judaicus). Signal peptides from different sources can be compared to identify a sequence that drives optimal levels of laccase secretion. For example, the native TXP-1 signal peptide and the signal peptides of D. melanogaster cuticle protein II and the tox21A may be combined.

In one embodiment the signal peptide is from larval cuticle protein 9.

In other embodiments the laccase is expressed intracellularly and in these embodiments the laccase in not fused to a signal peptide.

### Insects

One advantage of the present invention is that laccase may be expressed in insect species that can be easily cultivated on food or agricultural waste. Accordingly, any insect used for “bioconversion” of waste into insect biomass can be used to express laccase. Presently, only a few insect species are commercially used for insect-based bioconversion of food waste, with black soldier fly larvae (Hermetia illucens) being the most commonly used species. However, it is envisaged that any insect species amendable to genetic modification can be used to express laccase and form the basis of a laccase preparation. Considering the diversity of food and agricultural waste it is envisaged that waste-to-insect pairings to maximize both bioconversion and insect biomass (and hence laccase) production will be made by the skilled person.

For example, vegetative food wastes can be fed to both black soldier fly larvae and mealworm larvae, but this waste is too low in protein content for housefly larvae. Conversely, restaurant and kitchen wastes containing meat are well suited for housefly and black soldier fly larvae, but are too wet for mealworms, which can get moisture directly from the air and thus perform optimally in drier wastes. Further, black soldier fly larvae are tolerant of wet wastes and high temperatures (from bacterial and colony metabolism) allowing them to capitalize on many waste streams.

The choice of insect is based on a variety of factors including one or more of fast growth; high reproduction rates; high fecundity; large biomass; gregarious nature; short life cycle; disease resistant; ease of harvest; tolerate high stocking densities; have high food conversion rates; do not require excessive heating for reproduction and growth; and consume diets that are readily available, inexpensive to produce. In some embodiments the insects provide maximum dry yield. These characteristics ensure that the selected insects will rapidly increase in volume by both quantity and size. Further, the insect's rapid rate of growth and reproduction will contribute to the size of the harvest as well as provide replacement stock for a new crop of insects.

The insects may be cockroaches, flies, beetles, worms, larval stages of other flying insects such as meal worms, caterpillars, etc. The most desirable insects to produce laccase in terms of composition, size, reproduction, palatability, and lack of known toxins are typically species found within the orders Blattodea (cockroaches), Orthoptera (grasshoppers, locusts, katydids, crickets), Diptera (flies), and Lepidoptera (moths and butterflies)

It is envisaged that any dipteran insect may be used to express the laccase. Suitable dipteran insects include soldier flies, robber flies, bee flies, hover flies, fruit flies, vinegar flies, and blowflies.

In one embodiment, the insect is a fruit fly (Drosphila sp.), black soldier fly (Hermetia sp, for example Hermetia illucens), or house fly. Preferably the insect is a black soldier fly.

In other embodiments, the insect may be a mealworm, for example of the genus Tenebrio. In one embodiment the mealworm is Tenebrio molitor.

The laccase can be expressed in one or any combination of the eggs, larvae, pupae, and adults.

### Transformation of Insects

In general, transformation is a process in which exogenous DNA sequences are introduced into the insect germ line. Any laccase nucleic acid that can be integrated into the insect germ line can be utilized in accordance with the present invention. Numerous methods for transforming insects and nucleic acid vectors that can be used for insect transformation are known in the art.

To produce heritable changes the laccase or expression vector containing the laccase must be transformed into the insect during early development, prior to germ cell formation or directly within the germ line precursors. In flies, this can be achieved through three experimental approaches. First, editing tools are physically introduced via embryo transformation by microinjection, requiring outcrossing to ensure germ line transmission and identify unique events. Alternatively, in vivo remobilization allows gene targeting of complicated gene targeting constructs. Finally, in vivo upgrading allows novel material to be incorporated into previously established “docking” sites. The latter two are performed through genetic crosses conveniently circumventing any physical manipulation.

An initial step in making a transgenic insect typically involves embryo microinjection. Fertilized embryos are usually injected with a heterologous nucleic acid encoding a fungal laccase at the multinucleated syncytial one cell stage, just before cellularization to maximize the number of germ cells transformed. Injected insects are then outcrossed to identify germ line transmission of the nucleic acid.

Microinjection can be performed as “co-injection” or “direct” injections. During “co-injection” two components are introduced: the laccase nucleic acid, and a catalyst (e.g. recombinase, integrase, or nuclease). The catalyst can be provided in trans, as plasmid DNA encoding a promoter driving the catalyst, e.g., ΦC31 integrase, Cre recombinase, Flp recombinase, TALEN, or Cas9. Alternatively, the catalyst may be encoded by heterologous nucleic acid encoding a fungal laccase. In other embodiments, mRNA encoding the catalyst can be injected, i.e., ΦC31 integrase, Bxb1 integrase, ZFN, TALEN, or RGN1.

In some embodiments, transformation is performed using P-elements. A P-element is a transposon that is present in Drosophila melanogaster and is used widely for the creation of genetically modified flies. A P-element is a class II transposon, which means that its movement within the genome is made possible by a transposase. The complete element is 2907 bp and encodes a functional transposase.

Naturally-occurring P-elements typically contain a coding sequence for the transposase and recognition sequences for transposase action. The transposase catalyzes the excision of a P-element from the host DNA, cutting at two recognition sites, and then reinserts randomly. In general, to use P-elements as useful and controllable genetic tools, the two parts of a P-element are separated to prevent uncontrolled transposition. The normal genetic tools are, therefore, DNA coding for transposase with no transposase recognition sequences so it cannot insert, and a P-element construct. P-element constructs typically comprise a reporter useful for selecting transformants (e.g. white+, yellow+, etc.) and transposase recognition sequences. P-element constructs may further comprise a gene of interest, a bacterial reporter gene (e.g. gene encoding for antibiotic resistance), an origin of replication, etc.

In some embodiments, transformation is performed using piggyBac elements. A piggyBac element is a short inverted terminal repeat (ITR) transposable element that is approximately 2.5 kb long and comprises short (eg 13-bp) ITR sequences and an ORF. It is part of a subclass of ITR elements that insert exclusively into TTAA target sites. On insertion, the target site is duplicated with excision occurring in a precise fashion, restoring the insertion site. Beyond this functional similarity, the TTAA elements share no apparent structural identities. piggyBac vectors have been shown to mediate germ-line transformation in insect species.

A system involving Cre and FLP that allows for the study of two genes at identical places in the genome can also be used. In that system, an insect line is created by P-element insertion that contains the two transgenes of interest flanked by either loxP or FRT sequences. Under Cre expression, one transgene is removed, while under FLP expression, the other transgene is removed. Each remaining transgene is then left in the same chromosomal context.

In some embodiments, an approach to the site-specific integration problem is the use of homologous recombination. In general, the frequency of homologous recombination has been too low to be of practical use in insects. However, in some embodiments, the frequency of homologous recombination can be boosted by using P-element transformation to insert a construct containing the gene to be targeted, engineered with an I-Scel cutting site and flanked by two FRT sites. This construct can then be mobilized as a circular DNA molecule by expression of FLP and made linear by the expression of I-Scel, increasing the targeted recombination frequency. In this system, a separate P-element insertion carrying the homologous DNA engineered with I-Scel and FLP sites is required for each gene to be targeted. By this method, a targeted event could be obtained at a frequency of about 1 in 500-30,000 gametes from the female germline.

In some embodiments, the FLP/FRT system has been used to insert genes into any desired place in the genome. An integration frequency of up to 5% into a FRT site in the genome can be obtained when the target DNA is mobilized from elsewhere in the genome by FLP excision.

In some embodiments, transformation is performed using integrase-mediated systems. The site-specific integrase from phage φC31 has been shown to function at high frequency, requires no cofactors and mediates recombination between two sequences, the attB and attP sites, to create stable recombinants. Both intra- and inter-molecular recombination occur at high frequencies, and essentially no reversion of the reaction occurs. It has been demonstrated that the integrase can recognize and integrate into endogenous pseudo attP sites that have partial identity to attP.

In some embodiments, the φC31 integrase can mediate intra- and inter-molecular site-specific recombination at high frequency in insects. In some embodiments, transgenic insects can be created in attP-containing fly lines by integrating an attB-containing plasmid injected along with integrase mRNA into insect embryos.

### Laccase Preparations

In some embodiments, laccase preparations are produced from the insects. For example, the laccase preparations may be live or killed insects and comprise insect eggs, larvae, pupae, adults or any combination thereof.

Once the desired transgenic insect expressing the laccase is produced a quantity of adult or larval insects is introduced into a growth environment. The growth environment may be closed or partially closed. The growth environment preferably utilises a high-volume waste source such as fish and animal waste, processed or damaged fruit, vegetable, flowers grains, plant material, or other like food sources. Other waste sources include the by-products of a processing plant utilizing animal or vegetable components from such processes as farming operations, grain processing, fruit processing, or ethanol and bio-diesel production.

Once an insect population has reached the desired size and quantity to maximize biomass, the insects are then separated from the introduced food source during the harvesting process. In some embodiments, insect species (such as black solider fly) are chosen as the larvae have the ability to self-harvest. The harvesting process may include the use of vacuums, blower fans, washing stations, and screens/sieves to move and separate the insects.

The harvested insects are then dried, for example in commercial belt and tumble driers depending on the species and life stage (larval or adult). Once the insects have been desiccated, they may be ground into meal.

In one embodiment the laccase preparation consists of, or consists essentially of, insect meal.

The laccase preparation may further comprise an anti-caking agent, a preservative, a surfactant, and optionally a co-factor.

Suitable cofactors include a copper compound such as copper chloride, copper fluoride, copper benzoate dihydrate, and copper sulfate pentahydrate. Preferably the copper compound is copper sulfate pentahydrate.

In one embodiment the laccase preparation does not contain a mediator.

### Uses

Laccases exhibit broad substrate specificity and oxidize a broad range of contaminants including chlorinated phenolics, pesticides, and polycyclic aromatic hydrocarbons. Polycyclic aromatic hydrocarbons, which arise from natural oil deposits and utilization of fossil fuels are also degraded by laccases. In addition, laccases can oxidize carbozole, N-ethylcarbozole, fluorine, dibenzothiophene, phenols, aromatic amines and alkenes. Laccase can also immobilize soil pollutants by coupling them to soil humic substances, the contaminants that can be immobilized in this way include phenolic compounds including chlorinated phenols and anilines such as 3, 4-dichloroaniline, 2, 4, 6-trinitrotoluene, or chlorinated phenols. Thus, the laccase preparations described herein may be used in bioremediation of contaminated sites, or to process contaminated waste streams. In some embodiment the transgenic insects can be used to feed on contaminated waste.

The methods of using the laccase preparation (or the transgenic insects) involves contacting the preparation or insect with a substance containing a phenolic or aromatic contaminant. In embodiments using insects, the insect ingests the substance where it is expose to the expressed laccase.

In embodiment using a laccase preparation, the substance is incubated with the preparation for a period of time. Typically, the preparation is mixed into the substance (e.g. a contaminated liquid or wet waste) and incubated at ambient temperature for a period of time, for example at least an hour, 6 hours or overnight in order for the laccase in the preparation to act on the contaminant.

The laccase preparations can be used in the field of waste-water treatment. For example, the laccase preparations can be used in decolorization of colored compounds, in detoxification of phenolic components, and in in bioremediation. As described herein, the laccase preparations are capable of oxidizing a wide variety of compounds having different chemical structures, using oxygen as the electron acceptor. Accordingly, the laccases preparations described herein can be used in applications where it is desirable to remove one or a number of contaminants such as indigo carmine or BPA, in particular without the addition of a mediator or enhancer to the laccase preparation.

In some embodiments the laccase preparation reduces BPA concentration by about 5%, 10%, 15%, 20%, 25%, 30%, 35%, 40%, 45%, 50%, 55%, 56%, 60%, 65%, 70%, 75%, 80%, 85% or at least about 90%, for example compared to a control preparation not comprising a fungal laccase. In one embodiment the laccase preparation reduces BPA concentration by about 45%, 50%, 55%, 56%, 60%, or about 65%. In one embodiment the laccase preparation reduces BPA concentration by 50%, 55%, 56%, or about 60%.

The laccase preparations disclosed herein can be used presented herein to treat industrial wastewater. For example, the laccase preparations can be used in the treatment of liquid or wet waste from the textile industry such as bleaching or de-coloring indigo waste that is a by-product of denim manufacture.

The laccase preparations described herein can be used in the field of pulp and paper. For example, the laccase preparations can be used in the manufacture of paper pulps and fluff pulps from raw materials such as wood, bamboo, and cereal rice straw; the manufacture of paper and boards for printing and writing, packaging, sanitary and other technical uses; recycling of cellulose fiber for the purpose of making paper and boards; and the treatment of waste products generated by and treated at pulp or paper mills and other facilities specifically dedicated to the manufacture of paper, pulp, or fluff. The laccase preparations described herein can be useful, for example, in wood processing; in pulp bleaching; in wood fiber modification; in bio-glue (lignin activation) for MDF manufacturing; for enhanced paper properties; in ink removal; in paper dyeing; in adhesives (e.g. lignin based glue for particle- or fiber boards); etc.

The laccase preparations can be used as a feed additive or as part of a feed additive to increase the nutritional value of the feed for animals such as chickens, cows, pigs, goats, sheep, fish, etc or domestic pets. The laccase preparations may also be used as a processing aid to process plant materials and food industry by products with the aim to produce materials/products suitable as feed raw materials.

In some embodiments the laccase preparations and/or insects described herein can be used to reduce or remove contaminants such as indigo carmine and BPA.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### EXAMPLES

### Example 1: Transgenic D. melanogaster

Two strains of transgenic D. melanogaster were generated via standard embryo microinjection PhiC31 mediated integration methods. The transgenic strains expressed T. trogii laccse (SEQ ID NO: 1) modified as described above or Polyporus brumalis laccase as set out in SEQ ID NO: 3 with positions 1-66 replaced with a nucleic acid sequence coding for a Drosophila signal peptide as set out in SEQ ID NO: 3. SEQ ID NO: 3 is plasmid pMC-1-2-6 which comprises a sequence encoding the Drosophila larval cuticle protein 9 signal peptide from position 5795-5844 and the P. brumalis laccase from 5845-6980.

The endogenous signal peptides were replaced by the D. melanogaster larval cuticle protein 9, the sequence was codon optimized for D. melanogaster. Expression was driven by a short variant of the D. melanogaster tubulin promoter. The plasmids used to generate the transgenic flies are designated pMC-1-2-3 for the T. trogii laccse and pMC-1-2-6 and P. brumalis laccases, respectively. The sequence of pMC-1-2-3 is presented in SEQ ID NO: 3 and the sequence of pMC-1-2-6 is presented in SEQ ID NO: 4.

The laccase enzymes were codon optimised for expression in D. melanogaster using the IDT® codon optimisation tool. The native signal peptide was replaced with the D. melanogaster Larval Cuticle 9 signal peptide. gBlocks were generated with the D. melanogaster consensus Kozak sequence to drive efficient translation and assembled into the pMC1-1-1 expression vector (described below). Enzyme gene expression was under the control of the truncated α-tubulin promoter and an SV40 terminator. The truncated α-tubulin promoter was selected as a ubiquitous mid-level expression promoter.

Furthermore, its ubiquitous expression across many tissues with different physiological conditions makes it reasonable to expect that the enzyme will be expressed in favourable conditions for activity against the target pollutant.

For site specific chromosomal integration in D. melanogaster, the pMC-1-1-1 vector contains the PhiC31 AttB site for PhiC31 integrase mediated integration into D. melanogaster strains containing a PhiC31 AttP site. The PhiC31 mediated integration method was selected as these AttP sites reliably express transgenes, without unanticipated positional effects. To select for D. melanogaster transformed with laccase enzymes, the mini-white gene was used as a selectable marker. As plasmids containing this gene are injected into D. melanogaster strains that have a mutated white gene, the mini-white gene partially restores the red eye phenotype. These vectors were commercially microinjected into D. melanogaster embryos by BestGene Inc. The transformants were subsequently crossed the balancer strains to generate stable, homozygous, lines.

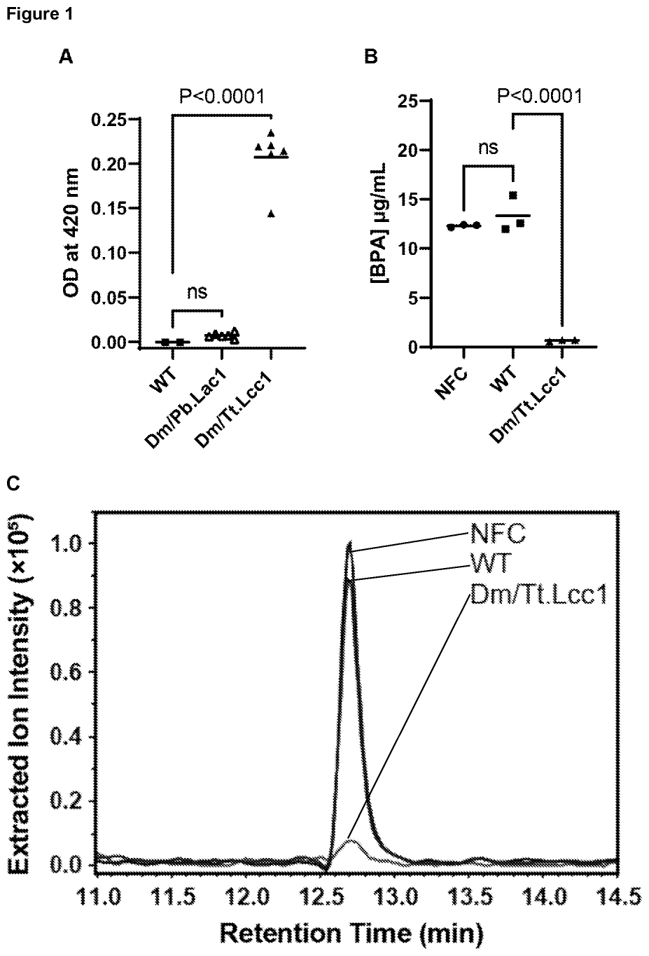

Laccase function was assessed by incubating fresh fly lysates in buffer containing ABTS, whose oxidation can be monitored colorimetrically at 420 nm. The results indicated that D. melanogaster expressing T. trogii laccase could oxidize ABTS, while neither D. melanogaster expressing P. brumalis laccase nor wild-type flies demonstrated this ability (FIG. 1A).

Lysate from flies expressing T. trogii laccase were also capable of degrading BPA (FIG. 1B).

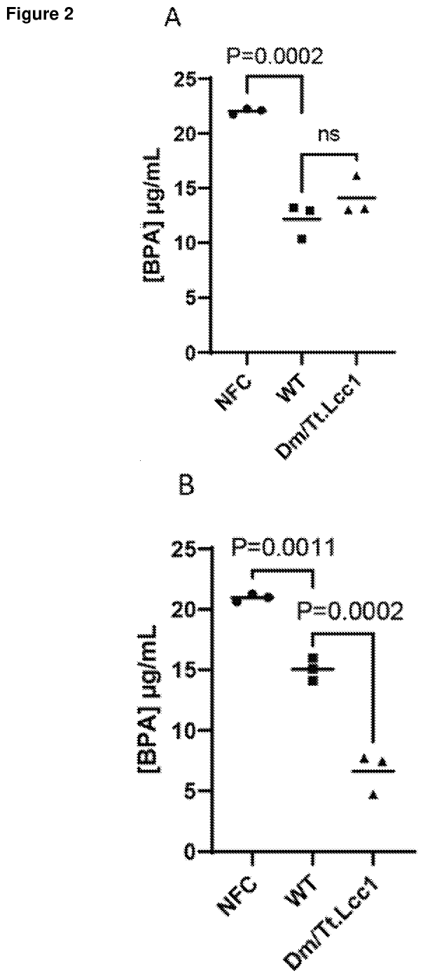

In vivo laccase activity was evaluated by rearing flies in media supplemented with ABTS. Flies expressing T. trogii laccase were capable of oxidizing ABTS in vivo, but wild-type flies did not have this activity (FIG. 2B).

In vivo degradation of BPA by T. trogii laccase expressing flies was dependent on the presence of ABTS (FIG. 2A without ABTS compared to FIG. 2B with ABTS).

Laccase activity of a powder generated from lyophilized flies was evaluated. Powder made from flies expressing T. trogii was compared to a commercially available purified laccase from Trametes versicolor (Table 1).

TABLE 1
Comparison of laccase activity in lyophilised
fly powder vs commercially available enzyme.

| Enzyme | Enzyme activity |
|---|---|
| Lyophilised D. melanogaster laccase | >1 U/g (total activity) |
| Commercial T. versicolor laccase | 75 U/g (specific activity) |

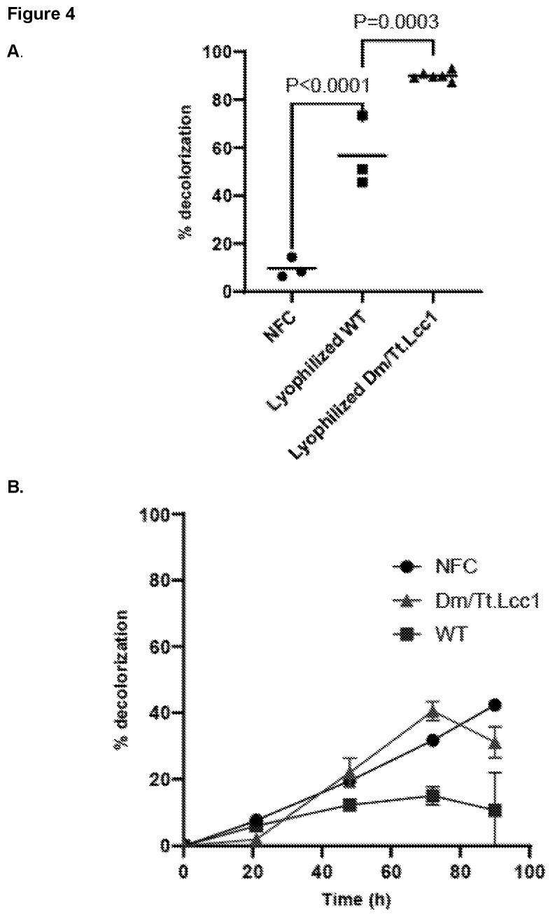

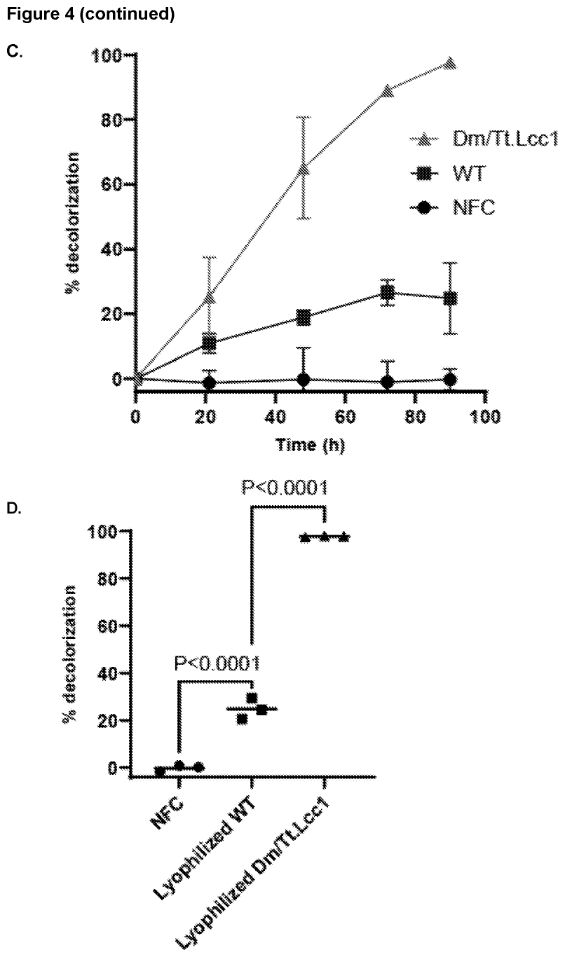

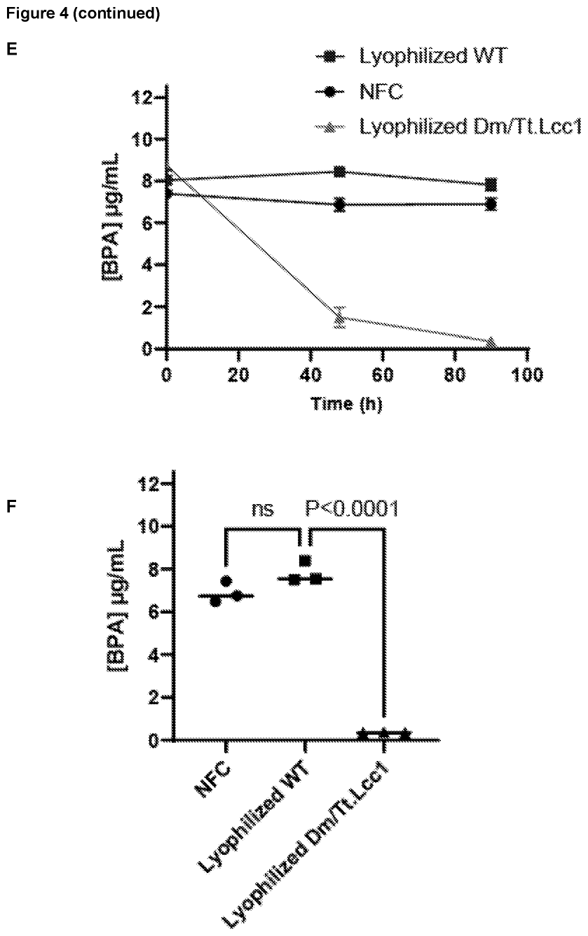

The lyophilized powder from T. trogii expressing flies could also oxidize BPA in the absence of a mediator (FIGS. 4E & 4F).

Incubating lyophilized powder with indigo carmine in water resulted in decolorization by both WT and transgenic powders (FIGS. 4A & 4B). This was presumed to be partially due to non-specific binding of the dye to insoluble debris. The inventors performed an assay of activity from clarified supernatant taken from the fly powder to distinguish between enzymatic decolourization and non-enzymatic binding. This required including copper, the laccase co-factor (not a mediator), however, robust decolorization was only observed from the T. trogii fly powder extract (FIGS. 4C & 4D).

The results demonstrate that transgenic insects may be used to bioremediate environmental contaminants in vivo and serve as production platforms for industrial enzymes.

### Example 2: Materials and Methods

### 2.01 the Enzyme Expression Plasmid

NEBuilder® HiFi DNA Assembly Master Mix (New England Biolabs) was used to assemble all constructs.

The pMC-1-1-1 vector was used to express the bioremediation enzymes in D. melanogaster. gBlocks encoding each enzyme were assembled (NEBuilder® HiFi DNA Assembly Master Mix) into pMC-1-1-1 linearised with NotI-HF (New England Biolabs). The constructed plasmids were verified by Sanger Sequencing. These gBlocks were generated from the laccase amino acid sequence, which were accessed from the GenBank. The enzyme sequences were codon optimised using the IDT® codon optimisation tool for expression in D. melanogaster. Sequences encoding native signal peptides were replaced with the D. melanogaster larval cuticle protein 9 signal peptide (Charles, et al (1998). Identification of proteins and developmental expression of RNAs encoded by the 65A cuticle protein gene cluster in Drosophila melanogaster. Insect Biochem. Mol. Biol. 28, 131-138). Immediately preceding the signal peptide was the D. melanogaster Kozak consensus sequence: AATCTTACAAA. Geneious (version 2020.1.2) was used to design and visualise the sequences.

pMBO2744 (Maselko, M. et al. Engineering multiple species-like genetic incompatibilities in insects. Nat. Commun. 11, 1-7 (2020)) was used as a backbone to generate the enzyme expression plasmids. A gBLOCK (IDT®) was synthesized that encodes a short alpha-tubulin promoter, a downstream SV40 polyadenylation sequence, and a NotI restriction site in between. This gBLOCK was installed into NotI linearized pMB02744 by HiFi assembly to produce pMC-1-1-1. This plasmid was then linearized with NotI, and gBLOCKs that encode for T. trogii laccase or P. brumalis laccase enzyme was installed with HiFi assembly to generate pMC-1-2-3 and pMC-1-2-6, respectively. The DNA sequences were codon optimized for expression in D. melanogaster using the IDT® codon optimization tool. The native fungal signal peptides were replaced with the larval cuticle protein 9 signal peptide. The Kozak sequence, AATCTTACAAA, was upstream of the start codon.

All plasmids were cloned and purified in E. coli Stbl3™ cells (Invitrogen, Competent Cell Selection Kit, A10469) made chemically competent using established procedures. E. coli strains were grown in LB medium supplemented with ampicillin (100 μg/mL, MP Biomedicals, CAS-No: 69-52-3). The plasmids were purified using the Monarch® Plasmid Miniprep Kit (New England Biolabs, #T1010L).

### 2.02 Fly Strains and Transgenesis

Canton S wild-type flies were from the Bloomington Drosophila Stock Centre (64349, RRID: BDSC 64349). To generate transgenic D. melanogaster, the enzyme expression plasmids were sent to BestGene Inc (Chino Hills, Ca) for plasmid DNA midiprep purification and microinjections. BestGene Inc performed φC31 integrase-mediated transformation and selected transformants, as indicated by an orange eye phenotype for chromosome 2 integration or a red eye phenotype for chromosome 3 integration. The inventors performed the crosses to appropriate balancer strains to generate stable transgenic lines. Heterozygous expression of all the laccases did not appear to affect survival.

Enzyme expression plasmids were purified using ZymoPURE™ II Plasmid Midiprep Kit (Zymo Research #D4200) and sent to BestGene Inc (Chino Hills, Ca) for φC31 mediated integration. All strains were maintained on a cornmeal diet based on the Bloomington standard Nutri-Fly formulation (catalog number 66-113; Genesee Scientific). In preparation for the in vitro or in vivo laccase enzyme activity assay, the diet for both the transgenic and wild-type strains was supplemented with CuSO₄·5H₂O (1 mM, Merck, CAS-no: 7758-99-9). Flies were reared in a controlled environment room at 25.0° C., 75% humidity, and a 12 hour light/dark cycle with a 30 minute transition period.

### 2.03 Fly Handling and Maintenance

Strains were maintained on a cornmeal diet based on the Bloomington Drosophila Stock Centre (BDSC) Cornmeal Food Recipe scaled to 500 mL. Stocks were either maintained in polystyrene narrow vials (Flystuff, #32-109RLBC) or polypropylene bottles (Flystuff, #32-130BF). Approximately every month, flies were transferred to fresh vials or bottles. In preparation for the laccase enzyme assays, the diet for both the transgenic and wild-type strains were supplemented with CuSO4·5H2O (1 mM, Merck, CAS-no: 7758-99-9), to facilitate laccase synthesis. Fly sorting was performed by briefly anaesthetising the flies with carbon dioxide and transferring them to a Flypad (Flystuff, Genesee Scientific) for selection with a microscope (Leica MZ 6). Flies were reared in a controlled environment room at 25° C., 60% humidity and a 12-hour light/dark cycle with a 30 minute transition period.

### 2.04 Laboratory Assays

### 2.04.1 Fly Lysate Preparation for In Vitro Enzyme Assays

To obtain a fly lysate, ten frozen adult flies were homogenized with a motorized pestle in the stated buffer supplemented with 10 mM CuSO₄·5H₂O and acid-washed glass beads. The samples were then agitated along a tube rack 10 times. Each tube was incubated for 15 minutes at 25° C. and centrifuged twice to remove insoluble debris.

Specifically, 100 μL of acid-washed glass beads (Sigma Aldrich, Catalogue #G8772) were added to 1.5 mL Eppendorf tubes (SSI bio). 10 adult flies were added into each tube and placed into a −30° C. freezer for at least 1 hour. 225 μL of 25 mM Acetate buffer pH 5 supplemented with 10 mM CuSO₄·5H₂O was added to each tube. The flies were homogenised with a motorised pestle (Kimble Kontes, Pellet Pestle Motor) and the tubes were run along a test tube rack 10 times. Each tube was incubated for 15 mins at 25° C. and then centrifuged (Thermo Scientific, Heraeus Pico 17 Microcentrifuge) at 17,000×g for 10 minutes. 100 μL of the supernatant containing soluble protein was transferred to a fresh tube and the centrifuge step was repeated. 81.5 μL of the supernatant was transferred to a fresh tube. The total protein content was measured in 1.5 μL with a NanoDrop (Thermo Scientific, NanoDrop Lite).

### 2.04.2 BPA In Vitro Enzyme Activity Assay Sample Preparation

A 100 mg/mL BPA stock was made by dissolving 1000 mg of BPA in 10 mL of methanol and mixing with a spatula until dissolved. From this, a 1 mg/mL BPA stock was made by diluting the 100 mg/mL 10-fold in methanol. A 0.25 mg/mL BPA stock was made by adding 250 μL of the 1 mg/mL stock with 750 μL methanol. The samples were prepared in triplicate by adding 10 μL of the 0.25 mg/mL BPA stock (final concentration 25 mg/L) to 80 uL of the fly lysate prepared in 4.2.1 and made up to 100 μL with 25 mM Acetate buffer pH 5 supplemented with 10 mM CuSO₄·5H₂O. Controls were prepared by adding 80 μL of wild-type fly lysate or 80 μL buffer for the NFC. The tubes were incubated at room temperature for approximately 24 hours. The reaction was stopped by quenching the reaction with 100 μL of 5% trichloroacetic acid (Sigma Aldrich, catalogue #T0699, CAS-no:76-03-9).

For the ABTS oxidation assay, fly lysates were prepared in citrate-phosphate buffer (pH 5) and added to 1 mM ABTS. The tubes were incubated at 25° C. for 2.5 hours. The reaction was quenched by adding 5% trichloroacetic acid (TCA, Sigma Aldrich, catalogue #T0699, CAS-no:76-03-9). A UV-Vis spectrophotometer (Jasco V-760 UV-Vis spectrophotometer) was used to measure the absorbance at 420 nm. The measurements were blanked to an equivalent sample that did not contain ABTS. For the BPA degradation assay, fly lysates or controls were prepared in sodium acetate buffer (pH 5) and added to 25 μg/mL BPA. The assay mixtures were incubated at room temperature for 21 hours. The reaction was quenched by adding 5% (v/v) trichloroacetic acid (Sigma Aldrich, catalog #T0699, CAS-no:76-03-9). BPA concentration was analyzed by LC-MS as described below.

### 2.04.3 BPA In Vivo Enzyme Activity Assay Sample Preparation

For the in vivo media, four conditions were made that contained (+) or did not contain (−) BPA or ABTS. These conditions were +BPA +ABTS, +BPA −ABTS, −BPA, +ABTS, and −BPA, −ABTS. All conditions consisted of the same minimal media components, which was made up of: 210 mL 25 mM sodium acetate buffer pH=5, 3.32 g yeast, 10.5 g sugar, and 1.38 g agar. The components were blended.

For the +BPA conditions, 100 mL of the minimal media was pipetted into a beaker and boiled in a microwave. 4 mL 1 M propionic acid pH 4.4 and 12.4 mg CuSO4·5H2O (1 mM final concentration) were added. Following this, BPA was added to a final concentration of 25 mg/L and stirred through. This media was then halved. One half was the +BPA −ABTS condition. To the other half 50 mg ABTS (1 mg/mL final concentration) was added to make the +BPA, +ABTS condition.

For the −BPA conditions, the remaining 100 mL of minimal media was pipetted into a beaker and boiled in a microwave. 4 mL 1 M propionic acid pH 4.4 and 12.4 mg CuSO4·5H2O (1 mM final concentration) was added. This media was halved. One half was the −BPA −ABTS condition. To the other half, 50 mg ABTS (1 mg/mL final concentration) was added to make the −BPA +ABTS condition.

For each condition, the hot liquid media (500 μL) was pipetted into 14 mL round bottom polypropylene culture tubes (Greiner, Z617954) and set overnight. 5 male and 5 female adult flies were added to the tubes, which were stoppered with cotton wool. The adults were incubated on the media for 3 days and then removed. After a total of 10 days the enzymatic reaction was stopped with 300 μL of TCA (5% v/v final concentration). The tubes were placed in a 100° C. water bath for 10 min. After complete melting of media, 500 μL of methanol (resulting in ˜1:1 methanol:TCA v/v) was added followed by vortexing. The tubes were then incubated in an orbital shaker/incubator (Ratek) at 300 rpm and 37° C. for 30 min. After incubation, the tubes were centrifuged at 4,347 rcf for 10 min at 20° C. The supernatant (700 μL) was transferred to an Eppendorf tube, which was centrifuged at 17,000 g for 10 min. This supernatant (600 μL) was syringe filtered (Filtropur S 0.2 μm, Sarstedt, 83.1826.001) for UHPLC-FLD analysis.

### 2.04.4 Lyophilisation

100-250 transgenic D. melanogaster or wild-type flies were added to a 1.5 mL Eppendorf tubes and placed in a −30° C. freezer for at least 1 hour. Tubes were placed upright into a freeze dryer (Alpha 1-4 LDplus, Christ) with the lid off and freeze dried for at least 40 hours at −45° C. at 0.1 mBar. The freeze-dried flies were weighed and then homogenised in a 20 mL porcelain mortar and pestle. The mortar and pestle were cleaned between samples by rinsing with tap water and then thoroughly cleaning with pyroneg. The pyroneg was rinsed off with Reverse Osmosis (RO) water and dried. The mortar and pestle were then sprayed down with 80% ethanol, washed off once more with RO water, and allowed to dry in a glassware oven for at least 15 minutes. The lyophilised samples were stored at 4° C.

### 2.04.5 Determining Specific Enzyme Activity and Total Enzyme Activity

The total activity of extracted lyophilised transgenic D. melanogaster was compared to the specific activity of dissolved commercial T. versicolor laccase. Activity assays were performed using an ABTS microplate assay (Greiner, 96-well microplates, #655903) on a PHERAStar FS plate reader (BMG Labtech). 5 mg of lyophilised transgenic D. melanogaster was resuspended with 500 μL of 25 mM sodium acetate buffer and 10 mM CuSO4·5H2O. The lyophilised samples were extracted at 25° C. for 15 minutes at 800 rpm and subsequently centrifuged for 10 minutes at 17,000 g. 400 μL of supernatant was retained and centrifuged again at 17,000 g for 10 minutes. 300 μL of supernatant was retained, and this was used as the neat transgenic D. melanogaster enzyme stock. To prepare the neat stock of the purified commercial T. versicolor laccase, 10 mg was dissolved in 20 mL of 25 mM sodium acetate buffer and 10 mM CuSO4·5H2O. The enzymes were serially diluted 1/10, 1/50, 1/100, 1/500, and 1/1000 in 25 mM sodium acetate buffer and 10 mM CuSO4·5H2O. The assay reaction mixture was composed of 80 μL of each enzyme dilution and 20 μL of 5 mM ABTS (1 mM ABTS final concentration). The path length was corrected to 1 cm using the PHERAStar plate reader path length correction setting for 100 μL volume and Greiner 96-well flat-bottomed microplates. ABTS oxidation was monitored at 420 nm every minute for 30 minutes. Enzyme activity was calculated from the enzyme dilutions that showed linear ABTS oxidation and absorbance readings within the dynamic range of the detector. One unit of enzyme was defined as the amount of enzyme that catalyses the oxidation of 1 μmol ABTS min⁻¹.

Volumetric enzyme activity (U/L) was calculated using the following equation:

Enzyme Activity (U/L)=ΔA·Vt·10⁶/ξ·l·Ve

Where ΔA is the rate of absorbance change (min⁻¹), Vt is the total reaction volume (0.1 L), ξ is the molar extinction coefficient (36000 L mol⁻¹ cm⁻¹ for ABTS119), Ve is the enzyme volume (0.08 L), and I is the path length (corrected to 1 cm).

The total activity of lyophilised transgenic D. melanogaster and specific activity of the purified commercial T. versicolor laccase (U mg⁻¹) was calculated by dividing the U L⁻¹ by the concentration of the lyophilised powder (mg L⁻¹).

2.04.6 Lyophilised Transgenic D. melanogaster Indigo Carmine Activity Assays

To test whether lyophilised transgenic D. melanogaster could be added directly to water, instead of buffer, to decolorise indigo carmine, 2.5 mg of lyophilised sample was added to 500 μL water. The sample was vortexed and 176 μL was added to 22 μL of 1 mg/mL indigo carmine (100 mg/L final concentration) and made up to 220 μL with water. The samples were incubated for 48 h at RT. At 0 hours and 48 hours, a 105 μL aliquot was centrifuged at 17,000 g for 5 minutes. 100 μL of supernatant was added to a microplate and read at 610 nm. Lyophilised wild-type or water only NFC served as controls.

As IC appeared to adsorb to the lyophilised powder, the lyophilised samples were extracted and tested for enzymatic activity in different buffer conditions. 2.5 mg of lyophilised sample was added to 500 μL of either water, 10 mM CuSO4·5H2O in water, 25 mM sodium acetate buffer pH 5, or 10 mM CuSO4·5H2O in 25 mM sodium acetate buffer pH 5. The lyophilised samples were extracted at 25° C. for 15 minutes at 800 rpm and subsequently centrifuged for 10 minutes at 17,000 g. 400 μL of supernatant was aliquoted and centrifuged again at 17,000 g for 10 minutes. 90 μL of sample or control (WT or NFC) was added to 10 μL 1 mg/mL indigo carmine (100 mg/L final concentration). An equivalent blank for each sample or control was prepared by adding 90 μL of the same sample to 10 μL water. The plate was incubated at RT with aeration for 90 hours. The samples were mixed and made up to 100 μL with water before reading at 610 nm at 0, 21, 48, 72, and 90 hours.

### 2.05 Detection of BPA by UHPLC Fluorescence Detection

### 2.05.1 Chemicals and Consumables

Bisphenol A (≥99%, 239659), methanol (HPLC grade, 34860), acetonitrile (HPLC grade, 34851), and potassium phosphate monobasic (ACS reagent, P0662-50G) were purchased from Sigma-Aldrich.

Water was purified using a Q-POD purification unit (Merck Millipore). Prior to analysis, all samples were centrifuge filtered (Amicon Ultra −0.5 mL Centrifugal Filters, Merck, UFC500396), standards and solutions were syringe filtered (Filtropur S 0.2 μm, Sarstedt, 83.1826.001), and the mobile phase solvents were vacuum pump filtered (0.22 μm JGWP Durapore membrane filters, Merck Millipore, GVWP04700).

### 2.05.2 UHPLC Analytical Method

The UHPLC analysis was performed on an Agilent 1290 Infinity LC System consisting of an Agilent 1290 Infinity Binary Pump (G4220A), Infinity autosampler and thermostat (G4226A, G1330B), column compartment (G1316C), and fluorescence detector (FLD) (G1321B). 30 μL of sample was loaded into 96-well plates (Corning 96 Well Clear Polystyrene Microplate, Merck, CLS3367) with a coverslip (Thermofisher Scientific, 15036), and kept at 10° C. before injection. The injection volume was 4 μL with a 5 sec needle wash at flush port for 5 sec, using acetonitrile. The separation was accomplished using an Agilent RRHD ZORBAX Eclipse Plus C18 3.0×150 1.8 μm (p/n 959759-302) supported with a UHPLC Guard Column, Eclipse Plus C18, 2.1 mm (p/n 821725-901). Data were processed with the Agilent 1290 UPLC OpenLAB CDS software (C.01.05 SP1 [49]). The isocratic mobile phase consisted of 50:50 v/v % ACN: 10 mM KH₂PO₄ and was set at a flow rate of 0.5 mL/min.

A gradient wash step was introduced after each run with eluent B 100% ACN and eluent A 10 mM KH₂PO₄, starting with 45% B1 from 0-1 min, increased to 70% B from 1-6 min, reduced to 5% B from 6-11 min, and finally 50% B from 11-16 min at 0.3 mL/min. BPA was detected using FLD set to an excitation wavelength of 229 nm, an emission wavelength of 316 nm, a gain setting of 12, and with an acquisition rate of 2.31 Hz.

### 2.05.3 Preparation of BPA Standards for UHPLC Analysis

For quantitating BPA in the in vitro BPA assays, a standard curve was prepared with BPA standards spiked into filtered wild type lysates at concentrations 25, 50, 250, 500, 2500, 5000 and 25,000 ng/mL. Then 40 μL of each standard was diluted 1:1 with 5% v/v TCA (2.5% v/v final concentration), to make the final BPA standard concentrations 12,500 ng/mL, 2,500 ng/mL, 1,250 ng/mL, 250 ng/mL, 125 ng/mL, 25 ng/mL, and 12.5 ng/mL.

To generate a standard curve that is representative of the in vivo sample matrix, the 12,500 ng/mL standard was made by adding 100 μL of the 25,000 ng/mL BPA stock made in methanol to 100 μL 5% v/v TCA. The following standards were made by serially diluting the 12,500 ng/mL stock in 50:50 methanol:TCA (2.5% v/v final concentration), to make the standards 2,500 ng/mL, 1,250 ng/mL, 250 ng/mL, 125 ng/mL, 25 ng/mL, and 12.5 ng/mL.

Linear regression of the BPA standard curves was used to calculate the calibration functions. The limit of detection (LOD) and limit of quantification (LOQ) were determined as the concentrations of BPA with a signal-to-noise ratio of approximately 3:1 and 10:1, respectively. The peak-to-peak method was used to determine noise.

### 2.06 Detection of BPA by Mass Spectrometry (MS)

### 2.06.1 BPA LC-MS Analytical Method

Degradation of BPA in the NFC, WT, and transgenic D. melanogaster samples were externally validated in triplicates by LC-MS at the Mass Spectrometry Facility at the University of Sydney. The analysis was performed on an Agilent 1100 Binary pump with autosampler, column heater and photo diode array (PDA), coupled to a Bruker amaZon SL mass spectrometer. Separation was accomplished using a Waters Sunfire C18 column (5 μm, 2.1 mm ID×15 mm). The mobile phase consisted of A Methanol, B Water with 0.1% acetic acid at a flow rate of 0.3 mL/min. The gradient consisted of 20% A from 0-9.3 minutes, followed by 76% A from 9.3-17.3 minutes, then 92% A from 17.3-26 minutes, and finally 20% A from 26-35 minutes. The elution of BPA was monitored by UV detection at wavelengths of 200 nm, 210 nm, 254 nm and 361 nm. Mass spectrometry (MS) was performed using Atmospheric Pressure Chemical Ionisation (ACPI) under positive and negative ionisation mode over a range of 200-1400 m/z. The operation parameters were: Nebulizer 27.3 psi, Dry Gas 4.0 L/min, Dry temperature 180° C., APCI Vaporisation temperature: 400° C., APCI Corona needle current: 6000 nA, Mass mode: enhanced resolution, SPS (Smart parameter setting) Target mass 150 m/z, compound stability 100%.

### 2.06.2 Preparation of BPA Standards for LC-MS Analysis

A 100,000 ng/mL BPA stock was prepared by dissolving 1.55 mg of BPA up to 15.5 mL in methanol. A 1,000 ng/mL stock was generated by diluting the 100,000 ng/mL BPA stock 100-fold in methanol. A 100,000 ng/mL internal standard BPA-D16 was prepared by adding 0.35 mg of Deuterated BPA-D16 to 3.5 mL of 100% methanol.

For the standard curve, the 20, 200, and 800 ng/mL standards were made by adding 20, 200, and 800 μL of the 1000 ng/mL BPA stock, respectively; to 25 μL of the 100,000 ng/mL internal standard BPA-D16 (2,500 ng/mL final concentration); and made up to 1000 μL with 100% methanol. The 2000, 5000, and 10,000 ng/mL standards were made by adding 20, 50, and 100 μL of the 100,000 ng/mL BPA stock, respectively; to 25 μL of the 100,000 ng/mL internal standard BPA-D16 (2,500 ng/mL final concentration); and made up to 1000 μL with 100% methanol. To calibrate how well BPA ionises in our sample matrix compared to the standard matrix, the BPA-D16 stock was diluted to 10,000 ng/mL and 20 μL was spiked into 60 μL of sample (2,500 ng/mL final concentration).

### 2.07 In Vivo Laccase Activity Assays

### 2.07.1 Minimal Media

The minimal media components were for 100 mL: 1.58 g yeast, 0.66 g agar, 5 g sugar, 4 mL 1 M propionic acid (pH 4.4), 12.4 mg CuSO4·5H2O (1 mM final concentration). A buffer of 20 mM citrate-phosphate (pH 5) was added for the initial ABTS assays, 25 mM sodium acetate buffer (pH 5) was used for all BPA experiments.

### 2.07.2 ABTS Only In Vivo Experiments

To minimal media 100 mg ABTS (1 mg/mL final concentration) was added. Three female and three male adult flies were incubated on the media for 48 hours, after which they were removed. For the all-male ABTS assay, the adult flies remained in the vial for 11 days. The appearance of the dark green radical cation was monitored and recorded with photographs.

### 2.07.3 ABTS+BPA Experiments

To minimal media, BPA was added (25 μg/mL final concentration). ABTS (1 mg/mL final concentration) was added for the +BPA, +ABTS condition.

For each condition, the liquid media was pipetted into a 14 mL polypropylene round-bottom culture tube. 5 male and 5 female adult flies were incubated on the media for 3 days and then removed. For the lysate condition, 50 μL of lysate from ˜100 flies was added to the tube. After a total of 10 days the enzymatic reaction was stopped with TCA (5% v/v final concentration). The media was melted in a water bath, after which methanol (making ˜1:1 methanol:aqueous TCA for a two-fold dilution of BPA) was added, followed by vortexing. The tubes were then incubated at 300 rpm and 37° C. for 30 minutes. After incubation, the tubes were centrifuged twice with the final supernatant reserved for BPA quantification by UHPLC-FLD as described below.

### 2.08 Lyophilized Dm/Tt.Lcc1 Enzyme Activity Assays

Frozen Dm/Tt.Lcc1 or wild-type flies were lyophilized (Alpha 1-4 LDplus, Christ) at −45° C. at 0.1 mBar. The freeze-dried flies were homogenized in a porcelain mortar and pestle and stored at 4° C.

For the activity assays, lyophilized Dm/Tt.Lcc1 and commercial T. versicolor laccase (Sigma-Aldrich, Catalogue 38429) were resuspended in 25 mM sodium acetate buffer (pH 5) supplemented with 10 mM CuSO4·5H2O. Lyophilized Dm/Tt.Lcc1 did not dissolve, and the soluble fraction was extracted at 25° C. for 15 minutes at 800 rpm and centrifuged twice to remove the insoluble debris. Enzyme serial dilutions were added in duplicate to 1 mM ABTS and ABTS oxidation was monitored on a PHERAStar FS plate reader (BMG Labtech) at 420 nm at 25° C. One unit of enzyme activity was defined as the amount of enzyme that catalyzes the oxidation of 1 μmol ABTS min-1 using the molar extinction coefficient 36 mM⁻¹ cm⁻¹ (Aslam, M. S., Aishy, A., Samra, Z. Q., Gull, I. & Athar, M. A. Identification, Purification and Characterization of a Novel Extracellular Laccase from Cladosporium Cladosporioides. Biotechnol. Biotechnol. Equip. 26, 3345-3350 (2012)).

For the indigo carmine decolorization assay, 5 mg/mL of lyophilized powder or controls in Millipore water were added to 100 mg/L indigo carmine (Sigma-Aldrich catalog 131164) and incubated at room temperature. At 0 and 48 hours, indigo carmine decolorization was monitored on a PHERAStar FS plate reader (BMG Labtech) at 610 nm.

As indigo carmine appeared to adsorb to the lyophilized powder, the soluble fraction was tested for enzymatic activity. 5 mg/mL lyophilized powder or controls in Millipore water supplemented with and without 10 mM CuSO₄·5H2O were extracted at 25° C. for 15 minutes at 800 rpm. The samples were centrifuged twice to remove the insoluble debris. The supernatant of the lyophilized powder and controls were added to 100 mg/L indigo carmine and incubated at room temperature. Indigo carmine decolorization was monitored on a PHERAStar FS plate reader (BMG Labtech) at 610 nm at 0, 21, 48, 72, and 90 hours.

For the BPA degradation assay, the soluble fraction of the lyophilized powder was extracted as described in the indigo carmine assay. The supernatant of the lyophilized powder and controls were added to 12.5 μg/mL BPA and incubated at room temperature. At 0, 48, and 96 hours, 100 μL aliquots were removed and the reaction was stopped using a 3 KDa nominal molecular weight limit (NMWL) centrifuge filter (Merck Amicon Ultra 0.5 mL Centrifugal Filters, UFC500396). BPA concentration was analyzed by UHPLC-FLD as described below.

### 2.09 BPA Quantification

BPA in samples was either quantified by UHPLC-FLD analysis or by LC-MS. UHPLC-FLD analysis was performed using an Agilent RRHD ZORBAX Eclipse Plus C18 column (3.0×150 mm 1.8 μm, p/n 959759-302) equipped with a UHPLC Eclipse Plus C18 Guard Column, (2.1 mm 1.8 μm, p/n 821725-901). The isocratic mobile phase consisted of acetonitrile (ACN):10 mM KH₂PO₄ (50:50 v/v) at a flow rate of 0.5 mL/min. A gradient wash step was introduced after each run with eluent B 100% ACN and eluent A 10 mM KH₂PO₄, starting with 45% B1 from 0-1 minute, 70% B from 1-6 minutes, 5% B from 6-11 minutes, and finally 50% B from 11-16 minutes at 0.3 mL/min. BPA was detected by setting FLD excitation wavelength at 229 nm, and emission wavelength at 316 nm and a gain setting of 12. Linear regression of 7-point standards from 12.5 ng/mL to 12,500 ng/mL BPA was performed to calibrate BPA concentration. All samples and standards were either filtered using syringe filters (Filtropur S 0.2 μm, Sarstedt, 83.1826.001) or centrifuge filters (Merck Amicon Ultra 0.5 mL Centrifugal Filters, UFC500396) prior to injection into the UHPLC.

LC-MS validation and quantification of BPA was performed externally at the Mass Spectrometry Facility at the University of Sydney. LC separation was performed using a Waters Sunfire C18 column (5 μm, 2.1 mm ID×15 mm) coupled to a Bruker amaZon SL mass spectrometer. Gradient elution was performed with a mobile phase consisting of (A) methanol and (B) water with 0.1% acetic acid at a flow rate of 0.3 mL/min. The gradient consisted of 20% A from 0-9.3 minutes, 76% A from 9.3-7.3 minutes, 92% A from 17.3-26 minutes, and finally 20% A from 26-35 minutes. MS analysis was performed using Atmospheric Pressure Chemical Ionisation (ACPI) under positive and negative ionization mode over a range of 200-1400 m/z. The operation parameters were: Nebulizer 27.3 psi, Dry Gas 4.0 L/min, Dry temperature 180° C., APCI Vaporisation temperature: 400° C., APCI Corona needle current: 6000 nA, Mass mode: enhanced resolution, SPS (Smart parameter setting) Target mass 150 m/z, compound stability 100%. Linear regression of 6-point BPA standards from 20 ng/mL to 10 μg/mL BPA was performed to calibrate BPA concentration. An internal deuterated BPA-D16 standard was spiked into the samples prior to injection to calibrate ionization in the sample matrix compared to the standard curve matrix prepared in methanol. All samples were centrifuge filtered (Merck Amicon Ultra 0.5 mL Centrifugal Filters, UFC500396) prior to injection into the LC-MS.

### 2.10 Statistical Analysis

Figures described herein were generated in Prism 8.4.2 or Prism 9 by GraphPad. For statistical analysis, a one-way ANOVA using Dunnet's method was conducted. The significance level for the analysis was 0.05, and all error bars represent the standard error of the mean. The exact P values, statistical tests used, and sample numbers (n) can be found in the figure legend.

### Example 3. Strains Plasmids and Primers

The following strains, plasmid and primers were used in the examples described herein

TABLE 2
D. melanogaster strains used herein

| Strain | Description | Reference | BDSC# |
|---|---|---|---|
| Wild Type | Canton S | Lindsley, et al | 64349 |
| | | Science 162, | |
| | | 993-993 (1968). | |
| 2nd Chr. attP3 | y¹ w¹¹¹⁸; PBac{y[+]- | Venken, et al | 9752 |
| | attP-3B}VK00037 | Science 314, | |
| | | 1747-1751 (2006). | |
| 2nd Chr. Bal, | w¹¹¹⁸; Gla/CyO; | This study | — |
| Tm2/Tm6b | Tm2/Tm6b | | |
| 2nd Chr. Bal, | w¹¹¹⁸; Bl/CyO; | This study | — |
| Tm2/Tm6b | Tm2/Tm6b | | |
| Dm/Tt.Lcc1 | y¹ w¹¹¹⁸; | This study | — |
| | PBac{Tt.Lcc1}attP-3B | | |
| Dm/Pb.Lac1 | y¹ w¹¹¹⁸; | This study | — |
| | PBac{Pb.Lac1}attP-3B | | |

TABLE 3
Plasmids used in this study

| | Integrated in | | |
|---|---|---|---|
| Plasmid | Fly Strain | Description | Reference |
| pMB02744 | NA | Contains a mini-white | Maselko et al. |
| | | gene for D. melanogaster | Nat. Commun. |
| | | selection, an AttB site | 11, 1-7 (2020). |
| | | for genomic integration, | |
| | | and an ampicillin | |
| | | resistance cassette for | |
| | | cloning in E. coli. | |
| pMC-1-1-1 | NA | Empty vector, with short | This study |
| | | alpha-tubulin promoter | |
| | | and SV40 regulatory | |
| | | sequences | |
| pMC-1-2-3 | Dm/Tt.Lcc1 | Short alpha-tubulin | This study |
| | | promoter driving | |
| | | expression of Lcc1 from | |
| | | Trametes trogii | |
| pMC-1-2-6 | Dm/Pb.Lac1 | Short alpha-tubulin | This study |
| | | promoter driving | |
| | | expression of Lac1 from | |
| | | Polyporus brumalis | |

TABLE 4
Primers

| Name | Sequence (5′ > 3′) | Notes | SEQ ID NO |
|---|---|---|---|
| 1stconstruct_F_2.0 | GCTTGGATTTCACTGGAACTAG | pMC-1-1-1 sequencing | 5 |
| 1stconstruct_R | TCGAGACCGTGACCTACATC | pMC-1-1-1 sequencing | 6 |
| 2ndconstruct_F_2.0 | CGTTTGTCAAGCCTCATAGC | pMC-1-2-3/pMC-1-2-6 | 7 |
| | | sequencing | |
| 2ndconstruct_R_2.0 | TTCATTTTATGTTTCAGGTTCAGGG | pMC-1-2-3/pMC-1-2-6 | 8 |
| | | sequencing | |
| P.bru_F | TGACTTTCAGGTGCCCGATC | pMC-1-2-6 sequencing | 9 |
| P.bru_R | GCCGGGAATGTGAGTTCGATG | pMC-1-2-6 sequencing | 10 |

3.1 gBlocks™ Used to Construct Expression Plasmids

gBlocks™ are double-stranded DNA fragments with sizes between used to facilitate the assembly of large and complex sequences. The following gBlocks were used in this study.

The First construct gBlock (SEQ ID NO: 11) includes short alpha-tubulin and SV40 regulatory sequences for expression of the enzymes in Drosophila melanogaster. Contains a NotI site between the promoter and terminator for assembly of enzyme gene gBlocks. Generates pMC-1-1-1 from pMBO2744

The ‘P. Bru Lac G’ gBlock (SEQ ID NO: 12) includes the Polyporus brumalis laccase gene, codon optimised for D. melanogaster with overlaps to pMC-1-1-1 and larval cuticle 9 signal peptide sequence. Generates pMC-1-2-6

The ‘T. tro. Lac’ gBlock (SEQ ID NO: 13) includes the Trametes trogii laccase gene, codon optimised for D. melanogaster with overlaps to pMC-1-1-1 and larval cuticle 9 signal peptide sequence. Generates pMC-1-2-3

### Example 4. Results

4.1 Screening for Fungal Laccase Activity in Transgenic D. melanogaster

D. melanogaster and other insects express endogenous laccases involved in immune defence. However, it was unknown if an insect could heterologously express a functional fungal laccase or if its expression would be toxic. We were also uncertain which tissues would provide the optimal expression of a functional laccase for either in vitro or in vivo activity. A short tubulin promoter was selected with the goal of achieving moderate expression across many tissues. The native fungal signal peptides were replaced with the D. melanogaster larval cuticle protein 9 signal peptide to facilitate extracellular secretion.

Homozygous transgenic strains were generated and expressed laccases from two fungal species, T. trogii and Polyporus brumalis, known to have a broad substrate range and high redox potential (Table 5). Fly lysates from both were assayed for oxidation of the chromogenic laccase substrate 2,2′-Azino-bis(3-ethylbenzthiazoline-6-sulfonic acid) (ABTS). The engineered D. melanogaster strain Dm/Tt.Lcc1, which expresses a laccase from T. trogii, oxidized ABTS (FIG. 1A). There was no activity observed in the wild-type or from the engineered Dm/Pb.Lac1 strain expressing a laccase from P. brumalis (FIG. 1A). Moving forward, we focused on characterizing Dm/Tt.Lcc1.

It was next determined if Dm/Tt.Lcc1 flies are capable of degrading bisphenol A (BPA), an endocrine disruptor and emerging environmental contaminant found in many consumer products. BPA has been shown to be a substrate of T. trogii laccase when expressed from its native host. We incubated lysates from flies with 25 μg/mL BPA for 21 hours and measured BPA concentrations by liquid-chromatography mass spectrometry (LC-MS). Lysates from Dm/Tt.Lcc1 reduced BPA concentrations by 95% compared to the no-fly controls and the wild-type fly lysate (FIGS. 1B, C). Overlaid representative extracted ion chromatograms (EIC, not shown) (m/z 227.15) illustrate that BPA is oxidised only by lysates from Dm/Tt.Lcc1.

TABLE 5
The fungal laccases engineered into D. melanogaster

| | | | Redox | |
|---|---|---|---|---|
| | | | potential | |
| Gene | | Genbank | (V vs. | Laccase |
| name | Native host | accession | NHE, pH 5) | substrates |
| Lcc1 | Trametes | AJ294820.1 | E° 0.79 V | Aminophenols, |
| | trogii | | | polyphenols, |
| | | | | industrial dyes |
| Lac1 | Polyporus | ABN13591.1 | E° 0.80 V | Industrial dyes, |
| | brumalis | | | aromatic |
| | | | | hydrocarbons |

### 4.2 In Vivo Laccase Activity

The potential of laccase expressing insects to oxidize substrates present in their environment by secreted enzymes was evaluated. Flies were reared on a minimal media to avoid binding of substrates to insoluble components present in standard media. The minimal media included 1 mM CuSO₄ to supply the laccase co-factor. First, 3 adult males and 3 adult females were reared on minimal diet including 1 mg/mL ABTS for 2 days, after which they were removed. Larvae 142 of the mated flies were grown for an additional 5 days. Dm/Tt.Lcc1 flies oxidized ABTS as indicated by blue colorization of the fly media, however, no oxidation was observed with wild-type flies. We controlled for the possibility that the ABTS oxidation was due to laccase that may have been leaking from dead larvae by incubating 6 male flies in ABTS media. After 11 days, only the Dm/Tt.Lcc1 males had oxidized ABTS. This result indicates that the oxidation was due to enzyme secreted into the media.

Next, we reared 5 male and 5 female flies in a minimal diet containing 25 μg/mL BPA. Adults were removed after three days. On day 10, BPA was extracted from their diet and quantified by Ultra-High-Performance Liquid Chromatography coupled with fluorescence detection (UHPLC-FLD). Both Dm/Tt.Lcc1 and wild-type flies had reduced BPA concentrations by 36% and 45%, respectively, compared to no-fly controls (FIG. 2A). There was no significant difference between Dm/Tt.Lcc1 and wild-type indicating that the reduction in BPA concentration was not due to the activity of the laccase transgene. The reduction in BPA concentration for wild-type flies relative to the no-fly control may be in part due to BPA absorbed by the adult flies which were removed after three days. Another possibility is that BPA detoxification pathways have been characterized in mammals 54 and it is possible D. melanogaster also contains endogenous pathways capable of detoxifying BPA.

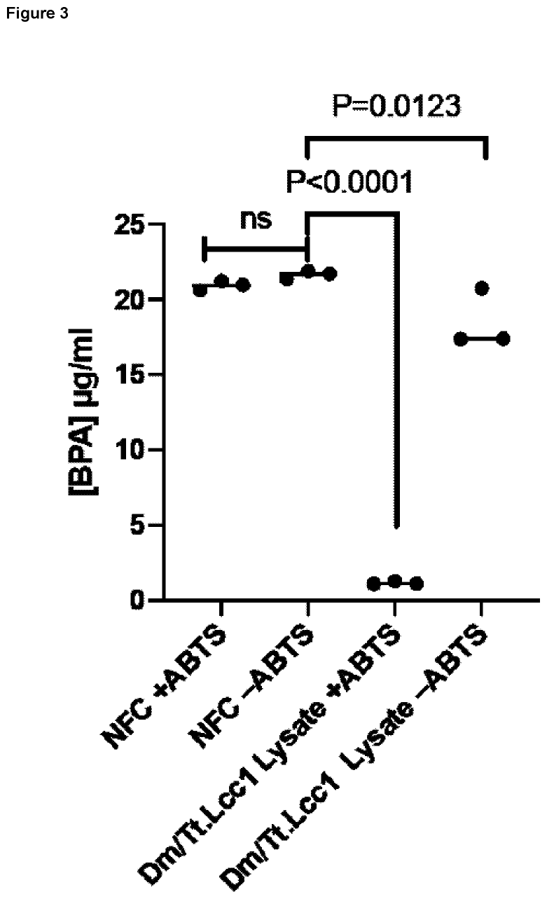

Some laccase substrates, such as ABTS, can function as redox mediators to facilitate the oxidation of additional compounds. When in vivo BPA degradation was tested in the presence of ABTS, Dm/Tt.Lcc1 flies effectively reduced BPA concentration by 68% and 56% compared to the no-fly control and wild-type, respectively (FIG. 2B). It is not clear why ABTS was necessary for the in vivo assay and not the in vitro lysate assays. Adding lysate directly to the media also required ABTS for efficient degredation, suggesting that there is some component of the diet which may be interfering (FIG. 3).

### 4.3 Lyophilized Insect Powder Laccase Activity

We next examined the potential of insects as platforms to produce a lyophilized laccase which can be easily stored and transported before use. A crude lyophilized powder from Dm/Tt.Lcc1 was compared to a commercially available purified laccase from Trametes versicolor using 178 ABTS as a substrate. The lyophilized Dm/Tt.Lcc1 displayed a total laccase activity of 1.4 U/g±179 0.1 SEM dry weight, while the commercial purified laccase displayed a specific activity of 75.3 180 U/g±6 SEM dry weight.

We next tested if Dm/Tt.Lcc1 lyophilized whole-fly powder had activity against indigo carmine, a textile industry dye and pollutant. Incubating 5 mg/mL of Dm/Tt.Lcc1 lyophilized powder in water containing 100 mg/L indigo carmine resulted in 90% decolorization after 48 h; however, WT fly lyophilized powder also decolorized 55% of the dye (FIG. 4A). We observed that insoluble material in the powder appeared to adsorb the dye. However, incubating dye using supernatant from centrifuged lyophilized Dm/Tt.Lcc1 in water did not appear to decolorize indigo carmine compared to controls (FIG. 4B). When the Cu²⁺ co-factor was included, supernatant from centrifuged lyophilized powder in water decolorized nearly 100% of indigo carmine in Dm/Tt.Lcc1 and 25% from WT flies after 90 hours (FIGS. 4C-D).

We also evaluated Dm/Tt.Lcc1 lyophilized powder activity against BPA. Incubating 12.5 μg/mL BPA with soluble enzyme from the lyophilized powder in the presence of Cu²⁺ co-factor resulted in a 96% reduction of BPA from Dm/Tt.Lcc1 fly powder after 90 hours. Incubation with WT fly powder resulted in a 3% reduction of BPA (FIGS. 4E-F).

### Example 5. Discussion

Engineering animals to heterologously express fungal/microbial enzymes may improve sustainable waste management, bioremediation, and facilitate the use of low-value waste streams as inputs for the production of high-value products such as industrial enzymes. Here, we engineer D. melanogaster to express a functional laccase from the fungus T. trogii. Lysates of this engineered fly (Dm/Tt.Lcc1) oxidized the endocrine disruptor BPA by 95% in vitro compared to the WT flies. Engineered flies also degraded BPA by 56% in vivo when added to their diet. Further, a powder prepared from lyophilized engineered flies also degraded BPA by 96% and the textile dye indigo carmine by almost 100% in vitro.

The findings presented herein demonstrate for the first time that insects can be stably engineered to express a functional fungal laccase, which demonstrated in vivo and in vitro laccase activity. Transgenic flies were capable of oxidizing laccase substrates in their environment and they can be used as a lyophilized powder to oxidize substrates in aqueous solutions.

1. A transgenic insect capable of expressing an active laccase, the insect comprising a heterologous nucleic acid encoding the laccase wherein the nucleic acid encoding the laccase is operably linked to a promoter.

2. The transgenic insect of claim 1, wherein the laccase is fungal laccase, preferably a T. trogii laccase.

3. The transgenic insect of claim 1, wherein the heterologous nucleic acid comprises SEQ ID NO: 1 or a sequence at least 80%, 85%, 90%, 95%, 97%, or 99% identical to SEQ ID NO: 1.

4. The transgenic insect of claim 1, wherein the laccase encoded by the heterologous nucleic acid is at least 80%, 85%, 90%, 95%, 97%, 99% or 100% identical to the laccase encoded by SEQ ID NO: 1.

5. The transgenic insect of claim 1, wherein the heterologous nucleic acid further comprises a sequence encoding a signal peptide in frame with the sequence encoding the laccase.

6. The transgenic insect of claim 5, wherein the signal peptide is larval cuticle protein 9 signal peptide, or a sequence at least 80%, 85%, 90%, 95%, 97%, or 99% identical to larval cuticle protein 9.

7. The transgenic insect of claim 1, wherein the promoter is the short tubulin alpha promoter.

8. The transgenic insect of claim 1, wherein the insect is selected from the genus Hermetia, Drosophila, or Tenebrio.

9. The transgenic insect of claim 8 wherein the insect is Hermetia illucens, Drosophila melanogaster, or Tenebrio molitor.

10. A laccase preparation comprising the transgenic insect of claim 1, or a portion thereof.

11. The laccase preparation of claim 10, wherein the transgenic insect is fully or partially dried.

12. The laccase preparation of claim 10, wherein transgenic insect is in the form of a meal or a powder.

13. The laccase preparation of claim 10, further comprising one or more of a preservative, anti-caking agent or surfactant.

14. The laccase preparation of claim 10, wherein the preparation does not comprise an added laccase mediator.

15. A method for removing or reducing the level of a phenolic or aromatic contaminant in a substance comprising contacting the substance with an effective amount of a laccase preparation of claim 10.

16. The method of claim 15 further comprising incubating the laccase preparation and the substance, preferably at ambient temperature.

17. (canceled)

18. The method of claim 15, wherein the substance is liquid waste or wet waste.

19. The method of claim 15, wherein the concentration of the phenolic or aromatic contaminant is reduced by about 5%, 10%, 15%, 20%, 25%, 30%, 35%, 40%, 45%, 50%, 55%, 56%, 60%, 65%, 70%, 75%, 80%, 85% or at least about 90%.

20. The method of claim 15, wherein the phenolic contaminant is BPA.

21. The method of claim 15, wherein the aromatic contaminant is an indigo dye.

22. A method for removing or reducing the level of a phenolic or aromatic contaminant in a substance comprising contacting the substance with the transgenic insect of claim 1 under conditions suitable for the insect to feed on the substance.
